(19) United States
(12) Patent Application Publication
(10) Pub. No.: US 2024/0417467 A1
(43) Pub. Date: Dec. 19, 2024

(54) Bispecific Engineered Antibodies

(71) Applicant: MedImmune, LLC, Gaithersburg, MD (US)

(72) Inventors: Yariv MAZOR, Gaithersburg, MD (US); Vaheh OGANESYAN, Gaithersburg, MD (US); Chi-I CHIANG, Gaithersburg, MD (US); John David BAGERT, Gaithersburg, MD (US); Xiuling LI, Gaithersburg, MD (US); Sterling PAYNE, Gaithersburg, MD (US); Even WALSENG, Gaithersburg, MD (US); Ying FU, Gaithersburg, MD (US); Chunlei WANG, Gaithersburg, MD (US); Chunning YANG, Gaithersburg, MD (US)

(21) Appl. No.: 18/628,697

(22) Filed: Apr. 6, 2024

Related U.S. Application Data
(60) Provisional application No. 63/494,905, filed on Apr. 7, 2023.

(51) Int. Cl. C07K 16/28

(52) U.S. Cl. CPC C07K 16/2809; C07K 16/2815; C07K 2317/31; C07K 2317/569

(57) ABSTRACT

Provided herein is a multispecific antibody containing two antigen binding arms capable of binding to a first target and at least one antigen binding arm capable of binding to a second target. The multispecific antibody includes a lambda charge pair introduced into the interface of a heavy chain and a light chain to reduce chain mispairing.

### CROSS-REFERENCE TO RELATED APPLICATIONS

This application claims the benefit of U.S. Provisional Patent Application No. 63/494,905, filed Apr. 7, 2023, which is incorporated herein by reference in its entirety.

### REFERENCE TO SEQUENCE LISTING SUBMITTED ELECTRONICALLY

The content of the electronically submitted sequence listing (Name: IOTS-101-US-NP Sequence Listing.xml; Size: 18,901 bytes; and Date of Creation: Apr. 2, 2024), filed with the application, is incorporated herein by reference in its entirety.

### FIELD OF THE DISCLOSURE

The present disclosure relates to multispecific antibodies containing two antigen binding arms capable of binding to a first target and one antigen binding arm capable of binding a second target, wherein the multispecific antibody includes a lambda charge pair introduced into the interface of a heavy chain and a light chain as a strategy for reducing chain mispairing. The disclosure also relates to methods of producing these multispecific antibodies and their therapeutic uses.

### BACKGROUND

Bispecific antibodies, which recognize two different epitopes, have become increasingly of interest in diagnostic and therapeutic applications and can support novel mechanisms of action that are not available to monospecific antibodies. However, their generation presents challenges. Promiscuous pairing of heavy and light chains of two antibodies expressed in one cell can result in the production of several different molecules, with only one being bispecific and the remaining pairings resulting in non-functional or monospecific molecules.

Various strategies have been developed as an attempt to overcome this problem and encourage the correct assembly of the desired bispecific. Such strategies for encouraging heterodimerization of two different heavy chains include techniques such as ‘knobs-into-hole’ (Ridgway 1990). Strategies for circumventing light chain mispairing include the use of a common light chain (Merchant 1998), domain swapping (Schaefer 2011), and replacement of a native disulfide bond with an inter-chain one (Mazor 2015).

An example of a bivalent bispecific format is a “DuetMab” described in WO 2013/096291. DuetMab antibody molecules uses knobs-into-holes technology for heterodimerization of two distinct heavy chains and increases the efficacy of cognate heavy and light chain pairing by replacing the native disulfide bond in one of the CH1-CL interfaces with an engineered disulfide bond. An example of an asymmetric trivalent bispecific format is the “2+1” format, where the bispecific antibody contains three antigen-binding arms, two of which bind to the same epitope, and the third binds to a different epitope.

While the above strategies have come some way to reduce chain mispairing, there remains a need for additional mechanisms to improve pairing of polypeptide chains in bispecific antibodies and facilitate their efficient production.

### SUMMARY OF THE DISCLOSURE

In some aspects, the disclosure involves improved pairing of chains in bispecific antibodies in a ‘2+1’ format, where the bispecific antibodies contain a first antigen binding arm that is capable of binding to a first epitope and two antigen binding arms (a second and a third antigen binding arm) that are capable of binding to a second epitope that is different to the first epitope. In this ‘2+1’ format, the third antigen binding arm is fused to the first or second antigen binding arm (e.g. via a peptide linker between domains in the respective heavy chain domains).

Improved pairing of heavy chain (HC) and light chains (LCs) could be achieved by introducing charge pairs into the interface a lambda LC and the HC. However, charge pairs designed for a kappa LC and CH1 interface were unlikely to be successful when applied to a lambda LC and CH1 interface. Amino acid residues at the interface between a lambda LC and HC where charge pairs could be introduced were identified, demonstrating that the introduction of these lambda charge pairs could advantageously improve chain pairing beyond what was achieved in the previous DuetMab bispecific format.

It was further established that the identified lambda charge pairs could be used to improve pairing in the ‘2+1’ format bispecific. As demonstrated herein 2+1 bispecific antibodies containing the lambda charge pairs were well expressed, showed high levels (>90%) of correct chain pairing and had good molecule stability. Furthermore, 2+1 bispecific antibodies containing the lambda charge pair and an antigen binding arm that targets CD3 on T-cells monovalently and another (non-T cell) target bivalently were demonstrated to elicit strong activity in a T-cell cytotoxicity assay, above what was achieved by a bispecific antibody that binds both targets monovalently. As such, in some aspects, only a single antigen binding domain binds to CD3 in the 2+1 bispecific antibody, and is referred to as a Bispecific T-cell Engager DuetMab (“TED2”).

Accordingly, in one aspect provided herein is a bispecific antibody comprising:
(a) a first antigen binding arm comprising a first light chain that is disulfide linked to a first heavy chain constant region 1 (CH1); and
(b) a second antigen binding arm comprising a second light chain that is disulfide linked to a second CH1,
(c) a third antigen binding arm comprising a third light chain that is disulfide linked to a third CH1,
wherein the first antigen binding arm binds to a first epitope, and the second and third antigen binding arms bind to a second epitope,
wherein the third antigen binding arm is fused to the first or second antigen binding arm,
wherein the first antigen binding arm, or the second and third antigen binding arms, comprise a lambda charge pair located at one or more of the following pairs of positions in a constant light chain lambda region (CLλ) of the light chain and the CH1:
(i) position 117 in the CLλ and position 141 in the CH1;
(ii) position 117 in the CLλ and position 185 in the CH1;
(iii) position 119 in the CLλ and position 128 in the CH1;
(iv) position 134 in the CLλ and position 128 in the CH1;
(v) position 134 in the CLλ and position 145 in the CH1;
(vi) position 134 in the CLλ and position 183 in the CH1;
(vii) position 136 in the CLλ and position 185 in the CH1;
(viii) position 178 in the CLλ and position 173 in the CH1; and
(ix) position 117 in the CLλ and position 187 in the CH1,
wherein the lambda charge pair comprises a positively charged amino acid residue selected from arginine, lysine or histidine located at one of the positions in the lambda charge pair and a negatively charged amino acid residue selected from aspartic acid, glutamic acid, serine or threonine located at the other position in the lambda charge pair, and
wherein the numbering is according to the EU index.

In some aspects, lambda charge pair is located at position 117 in the CLλ and position 141 in the CH1. In some aspects, the lambda charge pair is selected from the following list:
a. arginine at position 117 of the CLλ and aspartic acid at position 141 of the CH1;
b. arginine at position 117 of the CLλ and glutamic acid at position 141 of the CH1;
c. arginine at position 117 of the CLλ and serine at position 141 of the CH1;
d. arginine at position 117 of the CLλ and threonine at position 141 of the CH1;
e. lysine at position 117 of the CLλ and aspartic acid at position 141 of the CH1;
f. lysine at position 117 of the CLλ and glutamic acid at position 141 of the CH1;
g. lysine at position 117 of the CLλ and serine at position 141 of the CH1; and
h. lysine at position 117 of the CLλ and threonine at position 141 of the CH1.

In some aspects, the lambda charge pair is selected from a. to e. of the above list. In some aspects, the lambda charge pair is selected from any one of a., b., and e. of the above list. In some aspects, the lambda charge pair is selected from a. and b. of the above list. In some aspects, the lambda charge pair is arginine at position 117 of the CLλ and aspartic acid at position 141 of the CH1.

In some aspects, lambda charge pair is located at position 117 in the CLλ and position 185 in the CH1. In some aspects, the lambda charge pair is selected from the following list:
a. arginine at position 117 of the CLλ and aspartic acid at position 185 of the CH1;
b. arginine at position 117 of the CLλ and glutamic acid at position 185 of the CH1;
c. arginine at position 117 of the CLλ and serine at position 185 of the CH1;
d. arginine at position 117 of the CLλ and threonine at position 185 of the CH1;
e. lysine at position 117 of the CLλ and aspartic acid at position 185 of the CH1;
f. lysine at position 117 of the CLλ and glutamic acid at position 185 of the CH1;
g. lysine at position 117 of the CLλ and serine at position 185 of the CH1; and
h. lysine at position 117 of the CLλ and threonine at position 185 of CH1.

In some aspects, the lambda charge pair is located at position 119 in the CLλ and position 128 in the CH1. In some aspects, the lambda charge pair is selected from the following list:
a. arginine at position 119 of the CLλ and aspartic acid at position 128 of the CH1;
b. arginine at position 119 of the CLλ and glutamic acid at position 128 of the CH1;
c. arginine at position 119 of the CLλ and serine at position 128 of the CH1;
d. arginine at position 119 of the CLλ and threonine at position 128 of the CH1;
e. lysine at position 119 of the CLλ and aspartic acid at position 128 of the CH1;
f. lysine at position 119 of the CLλ and glutamic acid at position 128 of the CH1;
g. lysine at position 119 of the CLλ and serine at position 128 of the CH1; and
h. lysine at position 119 of the CLλ and threonine at position 128 of the CH1.

In some aspects, lambda charge pair is located at position 134 in the CLλ and position 128 in the CH1. In some aspects, the lambda charge pair is selected from the following list:
a. arginine at position 134 of the CLλ and aspartic acid at position 128 of the CH1;
b. arginine at position 134 of the CLλ and glutamic acid at position 128 of the CH1;
c. arginine at position 134 of the CLλ and serine at position 128 of the CH1;
d. arginine at position 134 of the CLλ and threonine at position 128 of the CH1;
e. lysine at position 134 of the CLλ and aspartic acid at position 128 of the CH1;
f. lysine at position 134 of the CLλ and glutamic acid at position 128 of the CH1;
g. lysine at position 134 of the CLλ and serine at position 128 of the CH1; and
h. lysine at position 134 of the CLλ and threonine at position 128 of the CH1.

In some aspects, lambda charge pair is located at position 134 in the CLλ and position 145 in the CH1. In some aspects, the lambda charge pair is selected from the following list:
a. arginine at position 134 of the CLλ and aspartic acid at position 145 of the CH1;
b. arginine at position 134 of the CLλ and glutamic acid at position 145 of the CH1;
c. arginine at position 134 of the CLλ and serine at position 145 of the CH1;
d. arginine at position 134 of the CLλ and threonine at position 145 of the CH1;
e. lysine at position 134 of the CLλ and aspartic acid at position 145 of the CH1;
f. lysine at position 134 of the CLλ and glutamic acid at position 145 of the CH1;
g. lysine at position 134 of the CLλ and serine at position 145 of the CH1; and
h. lysine at position 134 of the CLλ and threonine at position 145 of the CH1.

In some aspects, lambda charge pair is located at position 134 in the CLλ and position 183 in the CH1. In some aspects, the lambda charge pair is the lambda charge pair is a lysine at position 134 of the CLλ, and an aspartic acid or a serine at position 183 of the CH1.

In some aspects, lambda charge pair is located at position 136 in the CLλ and position 185 in the CH1. In some aspects, the lambda charge pair is selected from the following list:
a. arginine at position 136 of the CLλ and aspartic acid at position 185 of the CH1;
b. arginine at position 136 of the CLλ and glutamic acid at position 185 of the CH1;
c. arginine at position 136 of the CLλ and serine at position 185 of the CH1;
d. arginine at position 136 of the CLλ and threonine at position 185 of the CH1;
e. lysine at position 136 of the CLλ and aspartic acid at position 185 of the CH1;
f. lysine at position 136 of the CLλ and glutamic acid at position 185 of the CH1;
g. lysine at position 136 of the CLλ and serine at position 185 of the CH1; and
h. lysine at position 136 of the CLλ and threonine at position 185 of CH1.

In some aspects, lambda charge pair is located at position 178 in the CLλ and position 173 in the CH1. In some aspects, the lambda charge pair is selected from the following list:
a. arginine at position 178 of the CLλ and aspartic acid at position 173 of the CH1;
b. arginine at position 178 of the CLλ and glutamic acid at position 173 of the CH1;
c. arginine at position 178 of the CLλ and serine at position 173 of the CH1;
d. arginine at position 178 of the CLλ and threonine at position 173 of the CH1;
e. lysine at position 178 of the CLλ and aspartic acid at position 173 of the CH1;
f. lysine at position 178 of the CLλ and glutamic acid at position 173 of the CH1;
g. lysine at position 178 of the CLλ and serine at position 173 of the CH1; and
h. lysine at position 178 of the CLλ and threonine at position 173 of the CH1.

In some aspects, the first antigen binding arm comprises the lambda charge pair and the second and third antigen binding arms do not comprise the same lambda charge pair. In other aspects, the second and third antigen binding arms both comprise the same lambda charge pair and the first antigen binding arm does not comprise the same lambda charge pair.

As further described herein, the lambda charge pair can be combined with other approaches for encouraging light chain pairing, for example in order to further increase the correct assembly of the desired multispecific antibody.

In some aspects, the multispecific antibody has the native inter-chain disulfide bond in one of the CH1-CL interfaces replaced with an engineered inter-chain disulfide bond. In some aspects:
(i) the disulfide link between the first light chain and first CH1 is formed between a pair of cysteines engineered into the first light chain and first CH1, and the disulfide link between both the second light chain and second CH1, and third light chain and third CH1, is formed between a pair of native cysteines; or
(ii) the disulfide link between both the second light chain and second CH1, and third light chain and third CH1, is formed between a pair of cysteines engineered into both the second light chain and the second CH1, and third light chain and third CH1, and the disulfide link between the first light chain and first CH1 is formed between a pair of native cysteines.

In some aspects, the pair of cysteines engineered into the light chain and CH1 are located at position 122 of the light chain and position 126 of the CH1, and wherein the light chain comprises a non-cysteine residue at position 212 and the CH1 comprises a non-cysteine residue at position 220. In some aspects, the non-cysteine residues are valines.

In some aspects, the CLλ comprises an amino acid sequence having at least 90%, 91%, 92%, 93%, 94%, 95%, 96%, 97%, 98%, or 99% identity to SEQ ID NO: 1 or SEQ ID NO: 2. SEQ ID NO: 1 provides an exemplary wild type (native) CLλ, while SEQ ID NO: 2 provides an exemplary CLλ with the cysteine involved in the native inter-chain disulfide bond replaced with an engineered cysteine, for forming an engineered disulfide bond.

In some aspects, the CH1 comprises an amino acid sequence having at least 90%, 91%, 92%, 93%, 94%, 95%, 96%, 97%, 98%, or 99% identity to SEQ ID NO: 4 or SEQ ID NO: 5. SEQ ID NO: 4 provides an exemplary wild type (native) CH1, while SEQ ID NO: 5 provides an exemplary CH1 with the cysteine involved in the native inter-chain disulfide bond replaced with an engineered cysteine, for forming an engineered disulfide bond.

In some aspects, the light chain(s) in the antigen binding arm(s) that lacks the lambda charge pair comprises a constant light chain kappa region (CLκ). As described herein, the use of different light chains (lambda and kappa) is advantageous as it allows for methods such as light chain affinity chromatography to be used to selectively purify those multispecific antibodies containing the correct light chains. The inclusion of a kappa light chain in the multispecific antibody also allows for the inclusion of kappa charge pairs, which can encourage pairing of the second CH1:CLκ polypeptides.

In some aspects, the antigen binding arm(s) comprising the light chain with the CLκ comprises a kappa charge pair located in the CLκ and CH1 of the antigen binding arm(s), wherein the kappa charge pair comprises a positively charged amino acid residue selected from arginine, lysine or histidine located at one of the positions in the kappa charge pair and a negatively charged amino acid residue selected from aspartic acid, glutamic acid, serine or threonine located at the other position in the kappa charge pair.

In some aspects, the negatively charged amino acid residue in the kappa charge pair is at position 133 of the CLκ, and the positively charged amino acid residue in the kappa charge pair is at position 183 of the corresponding CH1 in that antigen binding arm. In some aspects, the negatively charged amino acid residue at position 133 of the CLκ is a glutamic acid, and wherein the positively charged amino acid residue at position 183 of the corresponding CH1 in that antigen binding arm is a lysine.

In some aspects, the CLκ comprises an amino acid sequence having at least 90%, 91%, 92%, 93%, 94%, 95%, 96%, 97%, 98%, or 99% identity to SEQ ID NO: 3.

In some aspects, the bispecific antibody is provided in a “2+2” format, comprising a fourth antigen binding arm, which comprises a fourth light chain that is disulfide linked to a fourth CH1. The fourth antigen binding arm binds to the same epitope as the first antigen binding arm, so the first and fourth antigen binding arms bind to a first epitope, and the second and third antigen binding arms bind to a second epitope. Thus in the 2+2 format, the bispecific antibody comprises two binding arms which bind to one epitope and two binding arms which bind to a different epitope. Like the first antigen binding arm, the fourth antigen binding arm comprises the lambda charge pair, i.e. both the first and fourth antigen binding arms comprise the lambda charge pair.

In some aspects, the second and third light chains are the same (i.e., they have identical amino acid sequences).

In some aspects, the first antigen binding arm and/or second antigen binding arm comprises an Fc region. In some aspects, the first antigen binding arm comprises a first Fc region and the second antigen binding arm comprises a second Fc region. Various strategies can be used to encourage heterodimerization of the two heavy chains (i.e. heterodimerization of a first heavy chain containing the first CH1 and first Fc region, and a second heavy chain containing the second CH1 and second Fc region).

In some aspects, the first and second Fc regions comprise modifications to facilitate heterodimerization of the first and second Fc regions. In some aspects, these modifications are located in the CH3 of the Fc regions.

In some aspects, the modification in the CH3 of one of first and second Fc regions is a substitution of an amino acid residue with one having a larger side chain, thereby generating a protuberance (knob) on the surface of said CH3 domain, and the modification in the CH3 of the other Fc region is a substitution of an amino acid residue with one having a smaller side chain, thereby generating a cavity (hole) on the surface of said CH3 domain, optionally wherein the CH3 domain containing the protuberance (knob) is part of the first heavy chain polypeptide and the CH3 domain containing the cavity (hole) is part of the second heavy chain.

In some aspects, wherein the substitution to generate a knob is a substitution to tryptophan at position 366 and the substitution to generate a hole is a substitution to generate a hole is one or more of the following:
i) a substitution to valine at position 407;
ii) a substitution to serine at position 366; and
iii) a substitution to alanine at position 368.

In some aspects, the CH3 domain containing the protuberance (knob) comprises a cysteine at position 354 and the CH3 domain containing the cavity (hole) comprises a cysteine at position 349.

In some aspects, the multispecific antibody comprises the lambda charge pairs in combination with any one or more of the engineered disulfides, kappa charge pairs and Fc modifications to facilitate heterodimerization described herein. For example, in some aspects, the multispecific antibody comprises the lambda charge pairs in combination with the engineered disulfides described herein. In some aspects, the multispecific antibody comprises the lambda charge pairs in combination with the kappa charge pairs described herein. In some aspects, the multispecific antibody comprises the lambda charge pairs in combination with the Fc modifications to facilitate heterodimerization described herein. In some aspects, the multispecific antibody comprises the lambda charge pairs in combination with engineered disulfides and Fc modifications to facilitate heterodimerization described herein. In some aspects, the multispecific antibody comprises the lambda charge pairs in combination with kappa charge pairs and Fc modifications to facilitate heterodimerization described herein. In some aspects, the multispecific antibody comprises the lambda charge pairs in combination with engineered disulfides and Fc modifications to facilitate heterodimerization described herein. In some aspects, the multispecific antibody comprises the lambda charge pairs in combination with engineered disulfides, kappa charge pairs and Fc modifications to facilitate heterodimerization described herein.

In some aspects, the first antigen binding arm binds to an epitope on CD3.

In some aspects, the multispecific antibody further comprising an additional antigen binding domain, optionally wherein the additional antigen binding domain is a VHH. The inclusion of an additional antigen binding domain in addition to the ‘2+1’ bispecific allows for the production of a trispecific antibody. Further, use of a VHH as one of the antigen binding domains reduces the number of heavy and light chains present during production of a trispecific antibody and therefore may be advantageous from a manufacturing standpoint as compared to including an additional antigen binding arm containing a further VH, VL and CH1.

In some aspects, the additional antigen binding domain (e.g. VHH) is capable of binding an epitope on CD8. In some aspects, the first antigen binding arm is capable of binding CD3 and the additional antigen binding domain (e.g. VHH) is capable of binding an epitope on CD8. Without wishing to be bound by theory, including an antigen binding domain (e.g. VHH) that is capable of binding CD8 in the multispecific is believed to allow for preferential activation of CD8⁺ T-cells, which may improve therapeutic efficacy.

As such, the format may be referred to as a Tetraspecific T-cell Engager DuetMab (“TED4”) and in some aspects comprises two antigen binding domains that bind the same target. In some aspects, the TED4 format comprises two antigen binding domains that bind the same or different antigen targets, a CD3 binding domain, and a CD8 binding domain.

In some aspects, one of the antigen binding arms of an Antigen 1/Antigen 2/CD3 TriMab is capable of binding to an epitope on Antigen 1 or Antigen 2 that does not induce cell cytotoxicity. In some aspects, Antigen 1 binding arm of an Antigen 1/Antigen 2/CD3 TriMab will induce cell cytotoxicity and Antigen 2 binding arm will act as an anchoring arm with no ability to induce cell cytotoxicity in cells expressing only Antigen 2. In some aspects, Antigen 2 binding arm of an Antigen 1/Antigen 2/CD3 TriMab will induce cell cytotoxicity and Antigen 1 binding arm will act as an anchoring arm with no ability to induce cell cytotoxicity in cells expressing only Antigen 1. The inability of the anchoring arm to induce cell cytotoxicity may be, for example, due to binding to a membrane distal epitope that prevents the ability to form an active immunological synapse.

Also provided herein is a method of producing the of producing the multispecific antibody described herein. In some aspects, the method comprises
a) expressing the first, second and third light chain and the first, second and third CH1 in a host cell;
b) allowing the first light chain to pair with the first CH1 so as to form the first binding arm, allowing the second light chain to pair with the second CH1 so as to form the second binding arm, allowing the third light chain to pair with the third CH1, and allowing the first binding arm to pair with the second binding arm so as to form the multispecific antibody; and
c) purifying the multispecific antibody from the host cell.

In some aspects, the method comprises producing the multispecific antibody, the method comprising expressing the first, second and third light chain and the first, second and third CH1 in a host cell; wherein the first light chain pairs with the first CH1 so as to form the first binding arm, wherein the second light chain pairs with the second CH1 so as to form the second binding arm, wherein the third light chain pairs with the third CH1, and wherein the first binding arm pairs with the second and the third binding arms so as to form the multispecific antibody; and purifying the multispecific antibody from the host cell.

In some aspects, purifying the multispecific antibody comprises affinity chromatography. In some aspects, the purifying the multispecific antibody comprises light chain affinity chromatography.

In some aspects, less than 25%, less than 20%, less than 15%, or less than 10%, less than 5%, less than 4%, less than 3%, less than 2%, or less than 1% of the light chains are mispaired following purification of the multispecific antibody. In some aspects, less than 10% of the light chains are mispaired. In some aspects, less than 5% of the light chains are mispaired. Suitable methods for determining the percentage of mispairing are described herein.

Also provided herein is one or more nucleic acid(s) encoding the multispecific antibody described herein. Also provided herein is an isolated host cell comprising the nucleic acid(s) or vector.

Also provided herein are pharmaceutical compositions and therapeutic methods involving the pharmaceutical compositions or multispecific antibodies, as further described below.

The disclosure includes the combination of the aspects and features described except where such a combination is clearly impermissible or expressly avoided.

### SUMMARY OF THE FIGURES

Aspects and experiments illustrating the principles of the disclosure will now be discussed with reference to the accompanying figures in which:

FIG. 1A illustrates the interface between a kappa light chain (LC) and CH1 of a heavy chain (HC) in an antibody. V133 of the kappa LC and S183 of the HC are labelled.

FIG. 1B illustrates the interface between a lambda LC and CH1 of a HC in an antibody. V134 and Y178 of the lambda LC and S183K of the HC are labelled.

FIG. 2 contains a schematic of the DuetMab antibodies containing charge pairs. The left hand “hole” HC is disulfide linked via native cysteines to a kappa LC and contains a kappa charge pair (e.g. S183K/V133E), indicated by the minus (“−”) symbol on the kappa LC and the plus (“+”) symbol on the “hole” HC. The right hand “knob” HC is disulfide bonded via engineered cysteines to the lambda LC and contains a lambda charge pair, indicated by the plus (“+”) symbol on the lambda LC and the minus (−) symbol on the “knob” HC.

FIG. 3 illustrates the interface between a lambda LC and CH1 of a HC in an antibody containing an exemplary lambda charge pair (T117R and A141S). T117R of the lambda LC and A141S of the HC are labelled.

FIG. 4. The data of % correct LC ratio in Table 1 were plotted in scatter X-Y chart. Charge pair variants #33, #34, #35, #36, and #41 were selected for additional analysis based on % correct LC ratio.

FIG. 5 shows the response signals and fitting curves of control sample #1 and variant #33, which is representative of the tested variants. Kinetics measurements to soluble monomeric form of Antigen 2 were obtained using an Ocet384 instrument. The dissociation constants, KD, were calculated as a ratio of k_{off}/kₒₙ from a non-linear fit of the data.

FIG. 6 shows DSC thermostability measurements captured transitions for the Fab, CH2, and CH3 domains under the T_{M1}, T_{M2}, T_{M3} and T_{M4} descriptions.

FIG. 7 shows UV chromatograms of sub-unit LC/MS analysis of each sample. No subunit corresponding to mispaired species was identified.

FIG. 8. Variants with different charge pairs were assayed for cytotoxic activity. Each point represents the mean values of triplicate wells and the ±standard error of the mean (SEM) is represented by error bars. R347 is isotype control.

FIG. 9 provides a representation of the data of % correct LC ratio in Table 9 plotted in grouped box chart.

FIG. 10 provides a cartoon representation of CH1-CL domain interface with mutations T117R in CL of lambda light chain and A141 D in the CH1 of heavy chain based on data generated from the crystallographic investigation described herein. A strong hydrogen bond with distance of approximately 2.4 Å appears formed between OD1 atom of aspartic acid at position 141 of CH1 domain and NH1 atom of arginine at position 117 of lambda light chain.

FIG. 11 provides a cartoon representation of CH1-CL domain interface with mutations T117R in CL of lambda light chain and A141 E in the CH1 of heavy chain based on data generated from the crystallographic investigation described herein. Hydrogen bond with distance of approximately 3.0 Å appears formed between OE1 atom of aspartic acid at position 141 of CH1 domain and NH1 atom of arginine at position 117 of lambda light chain.

FIG. 12 contains a schematic of the DuetMab ‘2+1’ antibodies containing charge pairs. The right hand “knob” HC is disulfide bonded via engineered cysteines to the lambda LC and contains a lambda charge pair, indicated by the plus (“+”) symbol on the lambda LC and the minus (“−”) symbol on the “knob” HC. The CH1 and VH region of this knob HC and lambda LC form a “first antigen binding arm”. The left hand “hole” HC is disulfide bonded via native cysteines to a kappa LC and contains a kappa charge pair (e.g. S183K/V133E), indicated by the minus (“−”) symbol on the kappa LC and the plus (“+”) symbol on the “hole” HC. The CH1 and VH region of this “hole” HC and kappa LC form a “second antigen binding arm”. A third antigen binding arm is fused from the N-terminus of its CH1 to the C-terminus of the “knob” HC by a peptide linker. The CH1 of the third antigen binding arm is disulfide bonded via native cysteines to a kappa LC and contains a kappa charge pair. As indicated by the different shading, the first antigen binding arm binds a first epitope (e.g., CD3) and the second and third binding arms bind a second, different epitope.

FIG. 13 provides representative images illustrating kinetics measurements to soluble monomeric form of Antigen 1 (top image) and heterodimeric form of CD3 epsilon/delta (bottom image) obtained using an Ocet384 instrument.

FIG. 14 provides UV chromatograms of sub-unit LC/MS analysis of the Antigen 1/CD3 2+1 bispecific (top chromatogram) and the NIP228/CD3 2+1 bispecific (bottom chromatogram). No subunit corresponding to mispaired species was identified.

FIG. 15 provides representative DSC thermostability measurement plots the of Antigen 1/CD3 2+1 bispecific (solid line) and the NIP228/CD3 2+1 bispecific (dotted line).

FIG. 16 provides killing assay and T cell activation (CD8 and CD4) data for DuetMabs and Duet2 (2+1) Bispecifics. Each point of cytotoxicity represents the mean values of triplicate wells and the ±standard error of the mean (SEM) is represented by error bars.

FIG. 17A and FIG. 17B contain a schematic of the DuetMab ‘2+2’ antibodies containing charge pairs. The antibodies correspond to those of FIG. 12, with an additional antigen binding arm. The antigen binding arms of matching colour bind the same epitope. FIG. 17A shows the symmetrical arrangement of the ‘2+2’ format, with antigen binding arms to the first epitope located at the N-termini of the heavy chains, and antigen binding arms to the second epitope immediately C-terminal to them. FIG. 17B shows the asymmetric arrangement of the ‘2+2’ format, with one heavy chain containing an antigen binding arm to the first epitope at its N-terminus and the other containing an antigen binding arm to the second epitope at its N-terminus.

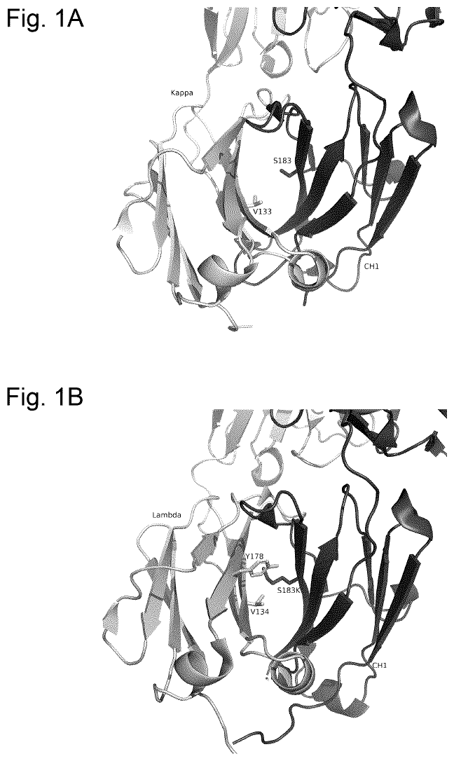

### DETAILED DESCRIPTION OF THE DISCLOSURE

Aspects and aspects of the present disclosure will now be discussed with reference to the accompanying figures. Further aspects and aspects will be apparent to those skilled in the art. All documents mentioned in this text are incorporated herein by reference.

Provided herein, as more fully discussed below, are multispecific antibodies (e.g. bispecific antibodies) in the ‘2+1’ format comprising lambda charge pairs located at certain pairs of positions in the constant light chain lambda region (CLλ) and a heavy chain constant region 1 (CH1) in one or more binding arms of the multispecific antibody. Also provided herein are multispecific antibodies that include a further antigen binding domain (e.g. a VHH) in addition to the antigen binding arms of the ‘2+1’ bispecific.

Methods are known for generating multispecific antibodies. Such methods, however, are often limited by a multitude of possible antibody formations which can include several combinations of incorrect pairings of heavy and light chains. Such mispairings can decrease production efficiency. The use of lambda charge variants as described herein overcome these limitations by preferentially causing the lambda light chain to pair with the correct CH1 in one binding arm, generating the preferred multispecific antibody assembly. In particular, these lambda charge variants can be combined with known approaches used to promote the correct pairing of heavy and light chains, such as Knobs into Holes (KiH), engineered disulfides and kappa charge pairing, as described in more detail below, to further improve the formation of the preferred multispecific antibody and reduce the formation of mispaired variants.

### Antibodies

The term “antibody” or “antibody molecule” describes an immunoglobulin whether natural or partly or wholly synthetically produced. The antibody may be human or humanized. In some aspects, the antibody is a monoclonal antibody molecule. Examples of antibodies are the immunoglobulin isotypes, such as immunoglobulin G (IgG), and their isotypic subclasses, such as IgG1, IgG2, IgG3 and IgG4, as well as fragments thereof.

An antibody is composed of two different types of polypeptide chain: one termed a heavy chain and the other terms a light chain. A natural monospecific antibody consists of two identical heavy chains and two identical light chains. The two heavy chains are linked to each other by disulfide bonds and each heavy chain is linked to a light chain by a disulfide bond. The disulfide bonds linking the light and heavy chains are sometimes termed “inter-chain” disulfide bonds, to distinguish them from the “intra-chain” disulfide bonds that are present within the individual heavy and light chain polypeptides.

Light chains in natural antibodies are either “lambda (A)” and kappa “(K)” light chains, which differ in terms of their amino acid sequence. Light chains are composed of a single constant light chain region (CL) and a single light chain variable region (VL). An example of a constant light chain lambda region (CLλ) amino acid sequence is provided as SEQ ID NO: 1 and an example of a constant light chain kappa region (CLκ) amino acid sequence is provided as SEQ ID NO: 3. Light chains used in the multispecific antibodies described herein may be chimeric light chains, e.g. contain a CLλ and a VLκ.

IgG heavy chains are composed of a heavy variable (VH) region and three heavy constant regions (CH1, CH2 and CH3), with an additional “hinge region” between CH1 and CH2. An example of an IgG1 CH1 region amino acid sequence is provided as SEQ ID NO:4. An example of an IgG1 CH2 amino acid sequence is provided as SEQ ID NO:6. An example of an IgG1 CH3 amino acid sequence is provided as SEQ ID NO: 7. An example of a heavy chain amino acid sequence comprising a CH1, hinge, CH2 and CH3 is provided as SEQ ID NO: 8.

Unless otherwise specified, amino acid residue positions in the constant domain, including the position of amino acid sequences, substitutions, deletions and insertions as described herein, are numbered according to EU numbering (Edelman, 2007).

The light chain associates with the VH and CH1 in the heavy chain to form an “antigen binding arm” and the variable domains in the antigen binding arm interact to form the “antigen binding domain”.

An “antigen binding domain” describes the part of a molecule that binds to all or part of the target epitope and generally comprises six complementarity-determining regions (CDRs); three in the VH region: HCDR1, HCDR2 and HCDR3, and three in the VL region: LCDR1, LCDR2, and LCDR3. The six CDRs together define the paratope of the antigen binding domain, which is the part of the antigen binding domain which binds to the target epitope. The term “epitope” as used herein refers to the part of an antigen that an antigen binding domain binds to. A monoclonal monospecific IgG antibody molecule contains two antigen binding domains, each of which are able to bind the same epitope (i.e. it is bivalent for a single epitope). The term “valent” as used herein notes the presence of specified number of antigen binding domains in the antibody that binds an epitope.

The VH region and VL region comprise framework regions (FRs) either side of each CDR, which provide a scaffold for the CDRs. From N-terminus to C-terminus, VH regions comprise the following structure: N term-[HFR1]-[HCDR1]-[HFR2]-[HCDR2]-[HFR3]-[HCDR3]-[HFR4]-C term; and VL regions comprise the following structure: N term-[LFR1]-[LCDR1]-[LFR2]-[LCDR2]-[LFR3]-[LCDR3]-[LFR4]-C term.

### Bispecific ‘2+1’ Antibodies

The present disclosure provides multispecific (e.g. bispecific) antibodies in the ‘2+1’ format. Multispecific antibodies according to the present disclosure may be provided in isolated form, in the sense of being free from contaminants, such as antibodies able to bind other polypeptides and/or serum components.

Multispecific antibodies of the present disclosure are capable of binding two different epitopes, either on the same or different antigens, and comprise three antigen binding arms, each of which is referred to herein as a “first antigen binding arm”, a “second antigen binding arm” and a “third antigen binding arm”. According to the present disclosure, an “antigen binding arm” comprises a light chain, a VH and CH1 (i.e. at least one constant and one variable domain of each of the heavy and light chain), where the light chain is disulfide linked to the CH1. Reference herein to a first, second and third domains (CH1, VH, VL, etc.) refers to the stated domain of the first, second and third antigen binding arm, respectively.

The formation of disulfide bonds between cysteine residues occurs during the folding of many proteins that enter the secretory pathway. As the polypeptide chain collapses, cysteines brought into proximity can form covalent linkages during a process catalyzed by members of the protein disulfide isomerase family. The term “disulfide link” or “disulfide linked” as used herein, refers to the single covalent bond formed from the coupling of thiol groups, especially of cysteine residues. In some aspects, the covalent linkage between two cysteines is between the two sulfur atoms of each residue. However, depending on the environment, not all protein species may have a disulfide present at all times, for example, in the event of disulfide reduction. Thus, the term “disulfide link” or “disulfide linked” (whether native or engineered), in some aspects, also refers to the presence of two cysteine residues that are capable of forming a disulfide link, irrespective of whether or not they are actually linked at that individual point in time.

In the ‘2+1’ format of the present multispecific antibodies, the first antigen binding arm binds a first epitope monovalently, and the second and third antigen binding arms bind to a second epitope bivalently, where the second epitope is different to the first epitope. The third antigen binding arm is fused to either the first or second antigen binding arm, typically via a peptide linker between the heavy chain domains. Suitable peptide linkers are known in the art and may consist of 5 to 100 amino acids, 5 to 50 amino acids, 5 to 25 amino acids, or 5 to 15 amino acids. Peptide linkers are formed mainly from glycine and serine amino acid residues and can comprise the amino acid sequences GGGGS (SEQ ID NO: 16) or SGGGGS (SEQ ID NO: 17). In one aspect, the peptide linker comprises or consists of (GGGGS)2 (SEQ ID NO: 18).

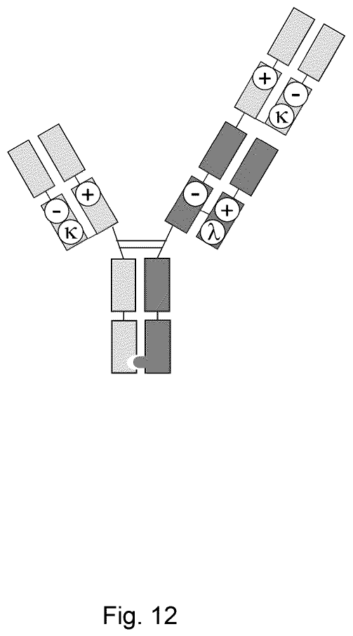

The third antigen binding arm is fused to one of the two heavy chains present in the multispecific antibody. In some aspects, the third antigen binding arm is fused at its N-terminus of the CH1 domain with the C-terminus of the VL domain on either the first antigen binding arm or the second antigen binding arm. Hence, in one aspect the N-terminus of the CH1 domain of the third antigen binding arm is fused to the C-terminus of the VL domain of the first antigen binding arm. A schematic of this exemplary aspect is provided in FIG. 12. In another aspect, the N-terminus of the CH1 domain of the third antigen binding arm is fused to the C-terminus of the VL domain of the second antigen binding arm.

The first and second antigen binding arms may further comprise additional heavy chain regions, i.e. one or more of the hinge, CH2 and CH3. In some aspects, the first and second antigen binding arm further comprises an Fc region (i.e. the remainder of the heavy chain comprising the hinge, CH2 and CH3). In some aspects, the first and second antigen binding arms comprise complete heavy chains (i.e. a VH, CH1, hinge, CH2 and CH3). In some aspects, the heavy chain of the first antigen binding arm is disulfide linked to the heavy chain of the second antigen binding arm (e.g. via inter-chain disulfide bonds between cysteines present in the Fc domains, or the capacity of those cysteines to form such bonds).

The first antigen binding arm differs from the second and third binding arms in at least one or more of the CDRs in the VL of the light chain and CH1 amino acid sequences, reflecting the fact that the first antigen binding arm binds a different epitope to that bound by the second and third antigen binding arms.

In some aspects, the second and third antigen binding arms have the same CDR sequences. In other aspects, they have the same VH and VL sequences. In some aspects, the second and third light chains are the same (i.e., they have identical amino acid sequences).

### Bispecific 2+2 Antibodies

Also provided are multispecific (e.g. bispecific) antibodies in the ‘2+2’ format, a variant of the ‘2+1’ format.

Essentially, a ‘2+2’ format multispecific antibody comprises the features of the ‘2+1’ format described above, plus a further, fourth antigen binding arm. By analogy to the first to third antigen binding arms described above, the fourth antigen binding arm comprises a fourth light chain disulfide-linked to a fourth CH1. The fourth antigen binding arm binds the same epitope as the first antigen binding arm (the “first epitope”, as referred to herein). Thus while the ‘2+1’ format antibodies described above bind the first epitope monovalently and the second epitope bivalently, the ‘2+2’ format antibodies bind each epitope divalently.

As in the ‘2+1’ format antibodies, the antigen binding arms which bind the same epitopes may be located on the same heavy chain or different heavy chains of the multispecific antibody. Typically, the four antigen binding arms are arranged such that each heavy chain of the antibody comprises one antigen binding arm that binds to the first epitope and one that binds to the second epitope. In these aspects the third antigen binding arm is fused to the first antigen binding arm and the fourth antigen binding arm is fused to the second antigen binding arm.

Alternatively, the antigen binding arms may be arranged such that each heavy chain of the antibody specifically binds one epitope, i.e. each heavy chain of the antibody comprises two antigen binding arms that bind the same epitope. Thus in some aspects the third antigen binding arm is fused to the second antigen binding arm and the fourth antigen binding arm is fused to the first antigen binding arm.

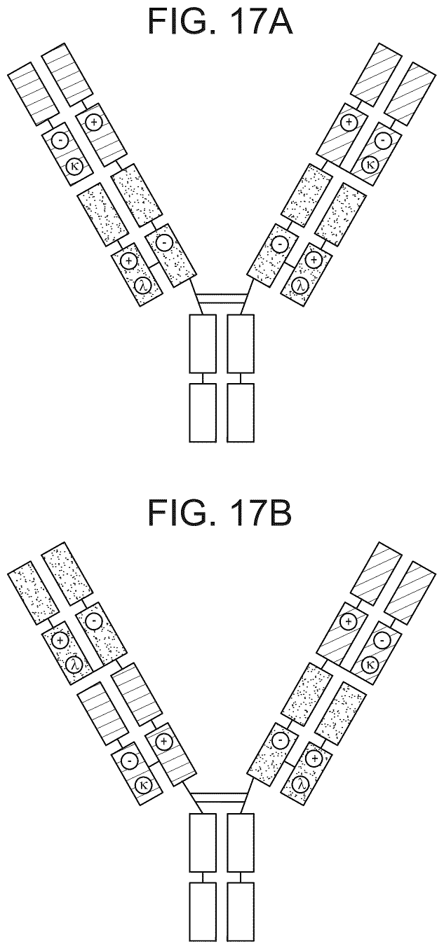

When the 2+2 format is arranged such that each heavy chain comprises one antigen binding arm that binds to the first epitope and one that binds to the second epitope, the antibody may have a symmetrical arrangement or an asymmetrical arrangement. The references to symmetry/asymmetry are in respect to the arrangement of the antigen binding arms within the two heavy chains of the antibody. Schematic diagrams of the symmetrical and asymmetrical arrangements are shown in FIGS. 17A and 17B, respectively.

In the symmetrical arrangement both antigen binding arms which recognize the first epitope are located either N-terminal or C-terminal to the antigen binding arms which recognize the second epitope. Thus in some aspects, the 2+2 format is arranged symmetrically, and the third antigen binding arm is fused to the N-terminus of the first antigen binding arm, and the second antigen binding arm is fused to the N-terminus of the fourth antigen binding arm. In other aspects, the 2+2 format is arranged symmetrically and the third antigen binding arm is fused to the C-terminus of the first antigen binding arm, and the second antigen binding arm is fused to the C-terminus of the fourth antigen binding arm.

In the asymmetrical arrangement, one antigen binding arm that binds the first epitope is N-terminal to an antigen binding arm that binds the second epitope, and the other antigen binding arm that binds the first epitope is C-terminal to an antigen binding arm that binds the second epitope. Thus in some aspects, the 2+2 format is arranged asymmetrically, such that the third antigen binding arm is fused to the N-terminus of the first antigen binding arm, and the second antigen binding arm is fused to the C-terminus of the fourth antigen binding arm (or vice versa).

As set out above, the first and second antigen binding arms may further comprise additional heavy chain regions, e.g. an Fc region. It will be appreciated from the foregoing discussion of the possible arrangements of the ‘2+2’ antibody format, that in this context, where additional heavy chain regions are present, an additional binding arm may be located between the first and/or second antigen binding arm and its respective additional heavy chain regions. For example, in some aspects the third antigen binding arm may be located between the first antigen binding arm and its additional heavy chain regions (e.g. Fc domain). In some aspects, the fourth antigen binding arm may be located between the second antigen binding arm and its additional heavy chain regions.

As set out above in respect of the second and third antigen binding arms, the first and fourth antigen binding arms may have the same CDR sequences. In some aspects they have the same VH and VL sequences. In some aspects, the first and fourth light chains are the same.

### Lambda Charge Pairs

The terms “charge pair(s)” and “charge mutation(s)” are used interchangeably throughout this specification and refer to a positively charged amino acid residue and a negatively charged amino acid residue, one of which is located in the a light chain region (e.g. constant light chain region) and the other in a heavy chain region (e.g. constant heavy chain region 1 (CH1)) of an antigen binding arm, located at positions intended to promote association of the light and heavy chains. By “lambda charge pair”, it is meant a charge pair where a positively or negatively charged amino acid residue is located in a lambda light chain (e.g. CLλ). By “kappa charge pair”, it is meant a charge pair where positively or negatively charged amino acid residue in the light chain is located in a kappa light chain (e.g. CLκ).

Without wishing to be bound by theory, it is believed that the oppositely charged amino acid residues in the charge pair increase the attraction of the heavy chain to the light chain in an antigen binding arm, thereby promoting formation of the antigen binding arm with the correct heavy and light chain.

At least one of the amino acid residues of the charge pair have been engineered into the antigen binding arm (i.e. at least one amino acid residue in the pair is not a wild-type amino acid residue). In some aspects, both amino acid residues in the charge pair are engineered into the antigen binding arm (i.e. both amino acid residues in the pair are not wild-type amino acid residues).

The amino acid residues of the charge pair are typically naturally occurring. Naturally occurring positively charged amino acid residues according to the present disclosure include arginine, lysine and histidine. Naturally occurring negatively charged amino acid residues according to the present disclosure include glutamic acid, serine, threonine and aspartic acid. Although serine and threonine are often described in the art as ‘uncharged’, they have an isoelectric point below 6 and therefore are partially negatively charged at neutral pH. For the purposes of the charge pairs disclosed herein, serine and threonine are examples of negatively charged amino acid residues (together with glutamic acid and aspartic acid).

Hence, a charge pair may comprise a positively charged amino acid residue selected from arginine, lysine or histidine located at one of the positions in the charge pair and a negatively charged amino acid residue selected from aspartic acid, glutamic acid, serine or threonine located at the other position in the charge pair. For example, the charge pair may comprise any one of the following pairs of amino acid residues:
arginine and aspartic acid;
arginine and glutamic acid;
arginine and serine;
arginine and threonine;
lysine and aspartic acid;
lysine and glutamic acid;
lysine and serine;
lysine and threonine;
histidine and aspartic acid;
histidine and glutamic acid;
histidine and serine; and
histidine and threonine.

In some aspects, the positively charged amino acid residue in the charge pair is located on the light chain and the negatively charged amino acid residue in the charge pair is located on the heavy chain. In other aspects, the negatively charged amino acid residue is located on the light chain and the positively charged amino acid residue in the charge pair is located on the heavy chain.

As exemplified herein, lambda charge pairs can be introduced at several positions to improve pairing of the correct light and heavy chains in the antigen binding arm.

In some aspects, the lambda charge pair comprises a positively or negatively charged amino acid residue at position 117, 119, 134, 136 or 178 of the constant light chain lambda region (CLλ). In some aspects, the lambda charge pair comprises a positively or negatively charged amino acid residue at position 141, 185, 128, 145, 183, 185, 173, or 187 of the CH1. As noted elsewhere, the numbering is according to EU numbering. Positions 117, 119, 134, 136, and 178 of the CLλ according to EU numbering corresponds to amino acid positions 10, 12, 27, 29, and 71 of SEQ ID Nos: 1 and 2. Positions 141, 185, 128, 145, 183, 185, 173, and 187 of the CH1 according to EU numbering corresponds to amino acid positions 24, 68, 11, 28, 66, 68, 56, and 70 of SEQ ID Nos: 4 and 5.

In some aspects, lambda charge pair located at one or more of the following pairs of positions:
(i) position 117 in the CLλ and position 141 in the CH1;
(ii) position 117 in the CLλ and position 185 in the CH1;
(iii) position 119 in the CLλ and position 128 in the CH1;
(iv) position 134 in the CLλ and position 128 in the CH1;
(v) position 134 in the CLλ and position 145 in the CH1;
(vi) position 134 in the CLλ and position 183 in the CH1;
(vii) position 136 in the CLλ and position 185 in the CH1;
(viii) position 178 in the CLλ and position 173 in the CH1; and
(ix) position 117 in the CLλ and position 187 in the CH1.

In some aspects, the lambda charge pair is located at charge pair is located at position 117 in the CLλ and position 141 in the CH1. For example, the lambda charge pair can be selected from the following list:
a. arginine at position 117 of the CLλ and aspartic acid at position 141 of the CH1;
b. arginine at position 117 of the CLλ and glutamic acid at position 141 of the CH1;
c. arginine at position 117 of the CLλ and serine at position 141 of the CH1;
d. arginine at position 117 of the CLλ and threonine at position 141 of the CH1;
e. lysine at position 117 of the CLλ and aspartic acid at position 141 of the CH1;
f. lysine at position 117 of the CLλ and glutamic acid at position 141 of the CH1;
g. lysine at position 117 of the CLλ and serine at position 141 of the CH1; and
h. lysine at position 117 of the CLλ and threonine at position 141 of the CH1.

In some aspects, the lambda charge pair is selected from any one of a. to f. of the above list. In some aspects, the lambda charge pair is selected from any one of a. to e. of the above list. In some aspects, the lambda charge pair is selected from any one of a., b., and e. of the above list. In some aspects, the lambda charge pair is a.

In some aspects, the lambda charge pair is located at charge pair is located at position 117 in the CLλ and position 185 in the CH1. For example, the lambda charge pair can be selected from the following list:
a. arginine at position 117 of the CLλ and aspartic acid at position 185 of the CH1;
b. arginine at position 117 of the CLλ and glutamic acid at position 185 of the CH1;
c. arginine at position 117 of the CLλ and serine at position 185 of the CH1;
d. arginine at position 117 of the CLλ and threonine at position 185 of the CH1;
e. lysine at position 117 of the CLλ and aspartic acid at position 185 of the CH1;
f. lysine at position 117 of the CLλ and glutamic acid at position 185 of the CH1;
g. lysine at position 117 of the CLλ and serine at position 185 of the CH1; and
h. lysine at position 117 of the CLλ and threonine at position 185 of the CH1.

In some aspects, the lambda charge pair is located at charge pair is located at position 119 in the CLλ and position 128 in the CH1. For example, the lambda charge pair can be selected from the following list:
a. arginine at position 119 of the CLλ and aspartic acid at position 128 of the CH1;
b. arginine at position 119 of the CLλ and glutamic acid at position 128 of the CH1;
c. arginine at position 119 of the CLλ and serine at position 128 of the CH1;
d. arginine at position 119 of the CLλ and threonine at position 128 of the CH1;
e. lysine at position 119 of the CLλ and aspartic acid at position 128 of the CH1;
f. lysine at position 119 of the CLλ and glutamic acid at position 128 of the CH1;
g. lysine at position 119 of the CLλ and serine at position 128 of the CH1; and
h. lysine at position 119 of the CLλ and threonine at position 128 of the CH1.

In some aspects, the lambda charge pair is located at charge pair is located at position 134 in the CLλ and position 128 in the CH1. For example, the lambda charge pair can be selected from the following list:
a. arginine at position 134 of the CLλ and aspartic acid at position 128 of the CH1;
b. arginine at position 134 of the CLλ and glutamic acid at position 128 of the CH1;
c. arginine at position 134 of the CLλ and serine at position 128 of the CH1;
d. arginine at position 134 of the CLλ and threonine at position 128 of the CH1;
e. lysine at position 134 of the CLλ and aspartic acid at position 128 of the CH1;
f. lysine at position 134 of the CLλ and glutamic acid at position 128 of the CH1;
g. lysine at position 134 of the CLλ and serine at position 128 of the CH1; and
h. lysine at position 134 of the CLλ and threonine at position 128 of the CH1.

In some aspects, the lambda charge pair is located at charge pair is located at position 134 in the CLλ and position 145 in the CH1. For example, the lambda charge pair can be selected from the following list:
a. arginine at position 134 of the CLλ and aspartic acid at position 145 of the CH1;
b. arginine at position 134 of the CLλ and glutamic acid at position 145 of the CH1;
c. arginine at position 134 of the CLλ and serine at position 145 of the CH1;
d. arginine at position 134 of the CLλ and threonine at position 145 of the CH1;
e. lysine at position 134 of the CLλ and aspartic acid at position 145 of the CH1;
f. lysine at position 134 of the CLλ and glutamic acid at position 145 of the CH1;
g. lysine at position 134 of the CLλ and serine at position 145 of the CH1; and
h. lysine at position 134 of the CLλ and threonine at position 145 of the CH1.

In some aspects, the lambda charge pair is located at charge pair is located at position 134 in the CLλ and position 183 in the CH1. For example, the lambda charge pair can be selected from the following list:
a. arginine at position 134 of the CLλ and aspartic acid at position 183 of the CH1;
b. arginine at position 134 of the CLλ and glutamic acid at position 183 of the CH1;
c. arginine at position 134 of the CLλ and serine at position 183 of the CH1;
d. arginine at position 134 of the CLλ and threonine at position 183 of the CH1;
e. lysine at position 134 of the CLλ and aspartic acid at position 183 of the CH1;
f. lysine at position 134 of the CLλ and glutamic acid at position 183 of the CH1;
g. lysine at position 134 of the CLλ and serine at position 183 of the CH1; and
h. lysine at position 134 of the CLλ and threonine at position 183 of the CH1.

In some aspects, the lambda charge pair is a lysine at position 134 of the CLλ, and an aspartic acid or a serine at position 183 of the CH1. In the CH1 sequences provided as SEQ ID NO: 4 or SEQ ID NO: 5, EU position 183 is a serine and therefore it is not necessary to introduce a modification in the CH1 of SEQ ID NO: 4 or SEQ ID NO: 5 in order to produce a charge pair with a positively charged amino acid at position 134 of the CLλ.

In some aspects, the lambda charge pair is located at charge pair is located at position 136 in the CLλ and position 185 in the CH1. For example, the lambda charge pair can be selected from the following list:
a. arginine at position 136 of the CLλ and aspartic acid at position 185 of the CH1;
b. arginine at position 136 of the CLλ and glutamic acid at position 185 of the CH1;
c. arginine at position 136 of the CLλ and serine at position 185 of the CH1;
d. arginine at position 136 of the CLλ and threonine at position 185 of the CH1;
e. lysine at position 136 of the CLλ and aspartic acid at position 185 of the CH1;
f. lysine at position 136 of the CLλ and glutamic acid at position 185 of the CH1;
g. lysine at position 136 of the CLλ and serine at position 185 of the CH1; and
h. lysine at position 136 of the CLλ and threonine at position 185 of the CH1.

In some aspects, the lambda charge pair is located at charge pair is located at position 178 in the CLλ and position 173 in the CH1. For example, the lambda charge pair can be selected from the following list:
a. arginine at position 178 of the CLλ and aspartic acid at position 173 of the CH1;
b. arginine at position 178 of the CLλ and glutamic acid at position 173 of the CH1;
c. arginine at position 178 of the CLλ and serine at position 173 of the CH1;
d. arginine at position 178 of the CLλ and threonine at position 173 of the CH1;
e. lysine at position 178 of the CLλ and aspartic acid at position 173 of the CH1;
f. lysine at position 178 of the CLλ and glutamic acid at position 173 of the CH1;
g. lysine at position 178 of the CLλ and serine at position 173 of the CH1; and
h. lysine at position 178 of the CLλ and threonine at position 173 of the CH1.

In some aspects, the antigen binding arm comprising a lambda charge pair comprises more than one lambda charge pair. For example, the first antigen binding arm may comprise two, three, four, five, six, seven, eight or nine lambda charge pairs at positions (i) to (ix) described above.

In the multispecific antibodies described herein, the lambda charge pair defined above is i) within the first antigen binding arm; or ii) within the second and third antigen binding arms, but the same lambda charge pair is not within all three antigen binding arms. In the context of the ‘2+2’ format antibodies described herein, the lambda charge pair defined above is i) within the first and fourth antigen binding arms; or ii) within the second and third antigen binding arms, but the same lambda charge pair is not within all four antigen binding arms.

Reference herein to the singular (e.g. “a” or “the”) charge pair or domain also encompasses multiple charge pairs or multiple domains, unless context clearly dictates otherwise. For example, as described herein, the first antigen binding arm, or both the second and third antigen binding arms, can comprise a lambda charge pair. In aspects where both the second and third antigen binding arms comprise a lambda charge pair, it is understood that the CH1 and CLλ of the second antigen binding arm comprises the lambda charge pair and also that the CH1 and CLλ of the third antigen binding arm comprises the same lambda charge pair.

In some aspects, the lambda charge pair is within the first antigen binding arm. That is, the first light chain comprises a CLλ and the lambda charge pair is between positions in the CLλ of the first light chain and the first CH1. In these aspects, the second and third antigen binding arms either contain a different lambda charge pair to that present in the first antigen binding arm, or do not contain a lambda charge pair (e.g., they comprise a wild type CLλ, or they comprise a constant light chain kappa region (CLκ)). In the context of ‘2+2’ format antibodies, in these aspects the lambda charge pair present in the first antigen binding arm is also present in the fourth antigen binding arm.

In some aspects, the lambda charge pair is within the second and third antigen binding arm. That is, the second and third light chains comprise a CLλ and the lambda charge pair is between positions in the CLλ of the second and third light chain and the second and third CH1. In these aspects, the first antigen binding arm (and where present, fourth antigen binding arm) either contains a different lambda charge pair to that present in the second and third antigen binding arms, or does not contain a lambda charge pair (e.g. it comprises a wild type CLλ, or it comprises a constant light chain kappa region (CLκ)).

For example, the first, second and third antigen binding arms in the multispecific antibody (and fourth antigen binding arm where present) may all comprise lambda charge pairs described above, wherein the lambda charge pair in the first antigen binding arm (and also in the fourth antigen binding arm, where present) is different to the lambda charge pair in the second and third antigen binding arms. That is, the lambda charge pair in the first antigen binding arm (and also in the fourth antigen binding arm, where present) may be located at any one of the pairs of positions at (i) to (ix) described above and the lambda charge pair in the second and third antigen binding arms located at a different pair of positions within (i) to (ix) described above. For example, the first antigen binding arm may comprise a lambda charge pair at position 117 in the CLλ of the first antigen binding arm and position 141 in the first CH1 and the second and third antigen binding arms may comprise a different lambda charge pair at e.g. position 134 in the CLλ of the second and third antigen binding arms and position 145 in the second and third CH1.

In another example, the first, second and third antigen binding arms in the multispecific antibody (and fourth antigen binding arm, where present) may all comprise a lambda charge pair at the same position (i.e. at one of (i) to (ix) described above), but the positively and negatively charged amino acid residues are on different polypeptide chains in the respective antigen binding arms. That is, the first antigen binding arm (and fourth antigen binding arm, where present) may comprise a positively charged amino acid residue at one of the positions in the CLλ and a negatively charged amino acid residue in the first CH1, and the second and third antigen binding arms comprise a negatively charged amino acid residue at the same position of the CLλ of the second and third antigen binding arms and a positively charged amino acid residue at the same position in the second and third CH1, or vice versa. For example, the first antigen binding arm may comprise a lambda charge pair that is an arginine at position 117 of the CLλ of the first antigen binding arm and aspartic acid at position 141 of the first CH1, and the second and third antigen binding arms may comprise a lambda charge pair that is aspartic acid at position 117 in the CLλ of the second and third antigen binding arm and arginine at position 141 of the second and third CH1.

In further examples, the first antigen binding arm (and fourth antigen binding arm, where present) comprises the lambda charge pair described above and the second and third antigen binding arms both comprise a constant light chain kappa region (CLκ), optionally with a kappa charge pair, as described in more detail below. Or the second and third antigen binding arms comprise the lambda charge pair described above and the first antigen binding arm (and fourth antigen binding arm, where present) comprises a constant light chain kappa region (CLκ), optionally with a kappa charge pair, as described in more detail below.

As demonstrated herein, multispecific antibodies containing lambda charge pairs exhibit improved correct light chain pairing when compared to multispecific antibodies lacking the lambda charge pair. That is, when producing the multispecific antibodies containing the lambda charge pair in the first antigen binding arm, the proportion of multispecific antibody containing the correct first light chain and first CH1 is increased compared to production of the equivalent multispecific antibody without the lambda charge pair.

As described in the examples, several methods are known that can be used to determine correct light chain paring. These include mass spectrometry-based approaches that can be used to establish association of the correct heavy/light chain. When multispecific antibody contains a mixture of kappa and lambda light chains, the ratio of kappa and lambda light chains in the assembled multispecific antibody can be determined using microfluidics-based electrophoresis as a readout of the correct light chain ratio.

Accordingly, in some aspects, the multispecific antibody containing the lambda charge pair exhibits improved correct light chain pairing when compared to an equivalent multispecific antibody that lacks the lambda charge pair. In some aspects, the multispecific antibody containing the lambda charge pair exhibits a correct light chain ratio greater than 90%, 95%, 96%, 97%, 98% or 99% (e.g. as determined using a microfluidics-based electrophoresis method), optionally after the multispecific antibody has been purified using light chain affinity purification.

Combination with Other Pairing Approaches

The lambda charge pairs described herein may be combined with other strategies for promoting heterodimerization in order to further increase the correct pairing of heavy and light chain polypeptides.

Non-limiting examples of strategies for promoting heterodimerization are described in more detail below and include using disulfide engineering at the CH1/CL interface, introducing additional charge pairs (e.g. kappa charge pairs) and Fc region modifications such as knobs-into-holes and allow fractionated purification strategies.

### Engineered Disulfides

In some aspects, the multispecific antibodies contain engineered disulfides in addition to the lambda charge pairs. By “engineered disulfides” it is meant that a native inter-chain disulfide bond at the CH1-CL interface (e.g. at 220 of the CH1 and 212 of the LC) of the first antigen binding arm, or second and third antibody binding arms has been replaced by an engineered (non-native) interchain disulfide, while the other antigen binding arm or arms contain the native interchain disulfide bond at the CH1-CL interface. An engineered disulfide is typically formed by engineering cysteines into the CL of a light chain and the CH1 of the corresponding heavy chain and replacing the cysteines that normally form the interchain disulfide. Disclosure related to the introduction of engineered disulfide into multispecific antibodies for the purpose of promoting heterodimerization can found e.g., in U.S. Pat. No. 9,527,927 and Mazor, 2015, which are herein incorporated by reference in their entirety.

Thus, in some aspects:
(i) the disulfide link between the first light chain and first CH1 is formed between a pair of cysteines engineered into the first light chain and the first CH1, and the disulfide links between the second light chain and second CH1, and third light chain and third CH1 are formed between pairs of native cysteines. A schematic of this aspect is provided in FIG. 12;
(ii) the disulfide links between the second light chain and second CH1, and third light chain and third CH1 are formed between pairs of cysteines engineered into the second and third light chains and second and third CH1s, and the disulfide link between the first light chain and first CH1 is formed between a pair of native cysteines; or
(iii) all three antigen binding arms contain engineered disulfides, but the cysteines engineered into the first antigen binding arm are at different positions (e.g. in the light chain) to the cysteines engineered into the second and third antigen binding arms.

In any of these three aspects, in the ‘2+2’ format the fourth antigen binding arm has the same type of disulfide link as the first antigen binding arm.

In some aspects, the pair of cysteines engineered into the CLλ and CH1 are located at position 122 of the CLλ and position 126 of the CH1, and wherein the same CLλ comprises a non-cysteine residue at position 212 and the same CH1 comprises a non-cysteine residue at position 220. In some aspects, the non-cysteine residues are valines.

An exemplary amino acid sequence of a CLλ comprising an engineered cysteine is provided as SEQ ID NO: 2 and an exemplary amino acid sequence of the CH1 comprising the corresponding engineered cysteine to form the engineered disulfide is provided as SEQ ID NO: 5.

In the bispecific antibodies exemplified herein, the engineered disulfide is present on the first (monovalent) antigen binding arm containing the lambda charge pairs and the native disulfide is present on the second (bivalent) and third antigen binding arms that do not contain the lambda charge pairs. However, other arrangements are also specifically contemplated, e.g., where the native disulfide is present on the antigen binding arm or arms containing the lambda charge pairs and the engineered disulfide is present on the arm or arms that lack the lambda charge pairs.

In some aspects, the pair of cysteines engineered into a constant light chain kappa region (CLκ) and CH1 are located at position 121 of the CLκ and position 126 of the CH1, and wherein the same CLκ comprises a non-cysteine residue at position 214 and the same CH1 comprises a non-cysteine residue at position 220. In some aspects, the non-cysteine residues are valines.

### Kappa Chain and Charge Pairs

In some aspects, at least one of the antigen binding arms comprises a light chain with a constant light chain kappa region (CLκ). That is, one of the antigen binding arms contains an CLλ and a different antigen binding arm comprises an CLκ in the multispecific antibody. As described herein, techniques such as light chain affinity chromatography that utilizes affinity resins specific for either CLκ or CLλ can be used to selectively purify antibodies based on their light chain. Examples of such affinity resins include the LambdaFabSelect and KappaSelect resins available from GE Healthcare. Such methods can be used to selectively purify multispecific antibodies containing both CLκ and CLλ and can therefore be used to improve production of multispecific antibodies in this format.

In one aspect, the first antigen binding arm (and fourth antigen binding arm, where present) comprises the lambda charge pair and the second and third antigen binding arms comprise a CLκ as part of the second and third light chain. In another aspect, the second and third antigen binding arms comprise the lambda charge pair and the first antigen binding arm (and fourth antigen binding arm, where present) comprises a CLκ as part of the first light chain.

An example of an CLκ amino acid sequence is provided as SEQ ID NO: 3.

In some aspects, the antigen binding arm(s) containing the CLκ comprises a kappa charge pair. As described above, kappa charge pairs refer to a positively charged amino acid residue and a negatively charged amino acid residue, one of which is located in the kappa light chain (e.g. CLκ) and the other in the heavy chain (e.g. CH1) of an antigen binding arm, located at positions intended to promote association of the light chain and CH1 of the second antigen binding arm.

In some aspects, the antigen binding arm(s) containing the CLκ comprises a kappa charge pair located at position 133 in the CLκ and position 183 in the second CH1. In some aspects, the negatively charged amino acid residue in the kappa charge pair is at position 133 of the CLκ and the positively charged amino acid residue in the kappa charge pair 183 of the second CH1. In other aspects, the positively charged amino acid residue in the kappa charge pair is at position 133 of the CLκ and the negatively charged amino acid residue in the kappa charge pair 183 of the second CH1. In some aspects, the negatively charged amino acid residue (e.g. at position 133 of the CLκ) is a glutamic acid, and wherein the positively charged amino acid residue (e.g. at position 183 of the second CH1) is a lysine. As noted elsewhere, this numbering is according to EU numbering.

Position 133 of the CLκ according to EU numbering corresponds to amino acid position 26 of SEQ ID NO: 3. Position 183 of the CH1 according to EU numbering corresponds to amino acid 66 of SEQ ID NOs: 4 and 5.

In certain exemplary aspects, the multispecific antibody comprises a first antigen binding arm (and optionally a fourth antigen binding arm) with a lambda charge pair as described above and a second and third antigen binding arm with a kappa charge pair as described above, wherein the multispecific antibody comprises engineered disulfides. Such exemplary aspects are illustrated in the schematics provided at FIGS. 12 and 17.

For example, in one exemplary aspect the first antigen binding arm (and optional fourth antigen binding arm) comprises a lambda charge pair (e.g. at position 117 in the CLλ and position 141 in the first CH1) and the disulfide link between the first light chain and first CH1 (and between the optional fourth light chain and fourth CH1) is formed between a pair of cysteines engineered into the CLλ and first CH1; and the second and third antigen binding arms comprise a kappa charge pair (e.g. at position 133 in the two CLκs and position 183 in the second and third CH1) and the disulfide links between the second light chain and second CH1, and third light chain and third CH1, is formed between pairs of native cysteines in the CLκ of the second and third light chain and second and third CH1.

Other combinations of kappa charge pairs and engineered disulfides are also specifically contemplated. In one such example, the first antigen binding arm (and optional fourth antigen binding arm) comprises the lambda charge pair and native disulfides, and the second and third antigen binding arms comprise a kappa charge pair and engineered disulfides. As another example, the first antigen binding arm (and optional fourth antigen binding arm) comprises a kappa charge pair and engineered disulfides, and the second and third antigen binding arms comprise the lambda charge pair and native disulfides. As yet another example, the first antigen binding arm (and optional fourth antigen binding arm) comprises a kappa charge pair and native disulfides, and the second and third antigen binding arms comprise the lambda charge pairs and engineered disulfides.

### Fc Region Modifications

As noted above, in some aspects the first and second antigen binding arms further comprise a first and second Fc region (i.e. further comprising the CH2 and CH3 regions of a heavy chain).

In some aspects, the multispecific antibodies comprise one or more modifications in one or more of the CH1, CH2 and CH3 domains that promotes formation of a heterodimeric antibody molecule by facilitating formation of the first and second Fc regions. This may involve a Knobs into Holes (KiH) strategy based on single amino acid substitutions in the CH3 domains that promote heavy chain heterodimerization as described in Ridgway, 1996. The knob variant heavy chain CH3 has a small amino acid has been replaced with a larger one, thereby generating a protuberance (knob) on the surface of said CH3 domain, and the hole variant has a large amino acid has replaced with a smaller one thereby generating a cavity (hole) on the surface of said CH3 domain. Additional modifications may also be introduced to stabilize the association between the heavy chains.

CH3 modifications to enhance heterodimerization include, for example, “hole” mutations Y407V/T366S/L368A on one Fc region and “knob” mutation T366W on the other Fc region. These may further include stabilizing cystine mutations Y349C (e.g. on the Fc region with the “hole” mutation) and stabilizing S354C mutation on the other Fc region (e.g. on the Fc region with the “knob” mutation”. Exemplary amino acid sequences of a CH3 domain engineered to contain a “hole” mutation are provided as SEQ ID NOs: 9 and 10 Exemplary amino acid sequences of a CH3 domain engineered to contain a “knob” mutation are provided as SEQ ID NOs: 11 and 12.

Accordingly, in one aspect, the substitution to generate a knob is a substitution to tryptophan at position 366 and the substitution to generate a hole is one or more of the following:
i) a substitution to valine at position 407;
ii) a substitution to serine at position 366; and
iii) a substitution to alanine at position 368.

In the multispecific antibodies exemplified herein, the “knob” is present on the first (monovalent) antigen binding arm and the hole is present on the second antigen binding arm (which, together with the third antigen binding arm, binds the second epitope bivalently). This arrangement is illustrated in the schematic provided as FIG. 12. However, the opposite arrangement is also specifically contemplated, i.e. where the “hole” is present on the CH3 of the first antigen binding arm and the “knob” is present on the CH3 of the second antigen binding arm.

Other examples of CH3 modification to enhance heterodimerization are described in, e.g. Table 1 of Brinkmann and Kontermann, 2017 MABS 9(2), 182-212, which is herein specifically incorporated by reference.

For example, the one Fc region may include a modification to allow fractionated elution by protein A chromatography as described in Tustian, 2016. Briefly, one of the Fc regions may comprise a modification that ablates binding to protein A (termed Fc*), allowing for selective purification of the heterodimeric FcFc* multispecific product. Examples of suitable modifications for generating an Fc* region include substitution of H435 with arginine and Y436 with phenylalanine.

In some aspects, the multispecific antibody comprises:
a first antigen binding arm comprising a lambda charge pair as described above and first Fc region;
a second antigen binding arm as described above comprising a second Fc region; and
a third antigen binding arm as described above,
wherein the first and second Fc regions comprise modifications to facilitate heterodimerization of the first and second Fc regions.

In some aspects, the multispecific antibody comprises:
a first antigen binding arm as described above comprising a first Fc region;
a second antigen binding arm comprising a lambda charge pair as described above comprising a second Fc region; and
a third antigen binding arm comprising the same lambda charge pair as the second antigen binding arm as described above,
wherein the first and second Fc regions comprise modifications to facilitate heterodimerization of the first and second Fc regions.

In some aspects, the multispecific antibody comprises:
a first antigen binding arm comprising a lambda charge pair as described above and a first Fc region;
a second antigen binding arm as described above comprising a second Fc region; and
a third antigen binding arm as described above,
wherein the first and second Fc regions comprise modifications to facilitate heterodimerization of the first and second Fc regions, and wherein the multispecific antibody comprises engineered disulfides.

In some aspects, the multispecific antibody comprises:
a first antigen binding arm as described above comprising a first Fc region;
a second antigen binding arm comprising a lambda charge pair as described above comprising a second Fc region; and
a third antigen binding arm comprising the same lambda charge pair as the second antigen binding arm as described above,
wherein the first and second Fc regions comprise modifications to facilitate heterodimerization of the first and second Fc regions, and wherein the multispecific antibody comprises engineered disulfides.

In some aspects, the multispecific antibody comprises:
a first antigen binding arm comprising a lambda charge pair as described above and a first Fc region;
a second antigen binding arm comprising a kappa charge pair as described above and a second Fc region;
a third antigen binding arm comprising the same kappa charge pair as the second antigen binding arm as described above,
wherein the first and second Fc regions comprise modifications to facilitate heterodimerization of the first and second Fc regions.

In some aspects, the multispecific antibody comprises:
a first antigen binding arm comprising a kappa charge pair as described above and a first Fc region;
a second antigen binding arm comprising a lambda charge pair as described above and a second Fc region;
a third antigen binding arm comprising the same lambda charge pair as the second antigen binding arm as described above,
wherein the first and second Fc regions comprise modifications to facilitate heterodimerization of the first and second Fc regions.

In some aspects, the multispecific antibody comprises:
a first antigen binding arm comprising a lambda charge pair as described above and a first Fc region;
a second antigen binding arm comprising a kappa charge pair as described above and a second Fc region; and
a third antigen binding arm comprising the same kappa charge pair as the second antigen binding arm as described above,
wherein the first and second Fc regions comprise modifications to facilitate heterodimerization of the first and second Fc regions, and wherein the multispecific antibody comprises engineered disulfides.

In some aspects, the multispecific antibody comprises:
a first antigen binding arm comprising a kappa charge pair as described above and a first Fc region;
a second antigen binding arm comprising a lambda charge pair as described above and a second Fc region; and
a third antigen binding arm comprising the same lambda charge pair as the second antigen binding arm as described above,
wherein the first and second Fc regions comprise modifications to facilitate heterodimerization of the first and second Fc regions, and wherein the multispecific antibody comprises engineered disulfides.

Non-limiting examples of multispecific antibodies comprising a lambda charge pair, a kappa charge pair, engineered disulfides and modifications to facilitate heterodimerization of the first and second Fc regions are provided in the examples.

Other Fc modifications contemplated herein are those that reduce or abrogate binding of the antibody molecule to one or more Fcγ receptors, such as FcγRI, FcγRIIa, FcγRIIb, FcγRIII and/or to complement. Such mutations reduce or abrogate Fc effector functions. Mutations that reduce or abrogate binding of an antibody molecule to one or more Fcγ receptors and/or complement are known and include the “triple mutation” or “TM” of L234F/L235E/P331S (according to European Union numbering convention) described for example in Organesyan et al., Acta Crystallogr D Biol Crystallogr 64(6): 700-704, 2008.

In some aspects, the CH2 domain of either or both immunoglobulin heavy chain constant domains comprises the following substitutions: E233P/L234V/L235A/G236del/S267K. This combination of mutations may be referred to herein as the “Fc effector null mutation”.

Other suitable Fc region amino acid substitutions or modifications are known in the art and include, for example, the triple substitution methionine (M) to tyrosine (Y) substitution in position 252, a serine (S) to threonine (T) substitution in position 254, and a threonine (T) to glutamic acid (E) substitution in position 256, numbered according to the EU index as in Rabat (M252Y/S254T/T256E; referred to as “YTE” or “YTE mutation”) (see, e.g., U.S. Pat. No. 7,658,921; U.S. Patent Application Publication 2014/0302058; and Yu et al., Antimicrob. Agents Chemother., 61(1): e01020-16 (2017), each of which is herein incorporated by reference in its entirety). This combination of mutations may extend the half-life of the antibody.

The triple mutation, Fc effector null mutation and YTE mutation, when present, may be present in one or both heavy chain constant domains. Typically, if included, they are included in both heavy chain constant domains.

In some aspects the Fc region comprises the YTE mutation and the triple mutation. In other aspects the Fc region comprises the YTE mutation and the Fc effector null mutation.

### CD3 Target and T-cell Engagers

In some aspects, the first antigen binding arm is capable of binding CD3.

CD3 (cluster of differentiation 3) is a protein complex composed of four subunits, the CD3γ chain, the CD3δ chain, and two CD3ε chains. CD3 associates with the T-cell receptor and the ζ chain to generate an activation signal in T lymphocytes. Bispecific antibodies that target CD3 and a target cell antigen have been used to force a temporary interaction between the target cell and T cell, causing cross-linking, T-cell activation, and subsequent antigen-dependent T cell killing of the target cell. The 2+1 format of bispecific antibodies is well-suited for CD3 binding as the goal is to bond only monovalently to the CD3 protein such that the T-cell receptor is only cross-linked and activated upon binding of the target cell.

### Additional Binding Domain

Also described herein are multispecific antibodies that further comprise an additional antigen binding domain that is capable of binding to a third epitope (e.g. a third epitope on a third antigen), which is different to the first and second epitope. Such antibodies may be a trispecific, tetravalent antibody: the first antigen binding arm binds a first epitope monovalently, and the second and third antigen binding arms bind to a second epitope bivalently, while the additional antigen binding domain binds to a third epitope monovalently. Alternatively such antibodies may be trispecific, pentavalent antibodies: the first and fourth antigen binding arms bind a first epitope bivalently, the second and third antigen binding arms bind a second epitope bivalently and the additional antigen binding domain binds to a third epitope monovalently.

In some aspects, the additional antigen binding domain is a single domain antibody, such as a heavy chain variable (VH) domain that lacks a CH1 and a light chain. The heavy-chain variable domain derived from a heavy-chain antibody that naturally lacks a light chain is referred to as VHH herein to distinguish it from the conventional VH of a four-chain immunoglobulin. This VHH molecule can be derived from antibodies produced camelidae species such as camels, alpacas, dromedaries, llamas, and guanaco. Species other camelidae can also produce heavy-chain antibodies that naturally lack a light chain, and such VHHs are also encompassed.

A VHH of camelidae heavy chain antibody can be obtained by genetic engineering process. See U.S. Pat. No. 5,759,808. Similar with other non-human antibody fragments, the amino acid sequence of the camelidae VHH can be altered recombinantly to obtain a sequence that more closely mimics a human sequence, i.e., “humanized”, thereby reducing the antigenicity of the Camelidae VHH to humans. In addition, key elements derived from the camelidae VHH can also be transferred to the human VH domain to obtain a camelized human VH domain.

The VHH has a molecular weight that is one-tenth the molecular weight of human IgG molecule, and has a physical diameter of only a few nanometers. The VHH itself has extremely high thermal stability, stability to extreme pH and proteolytic digestion, and low antigenicity. Making use of a VHH as one of the antigen binding domains reduces the number of heavy and light chains present during production of a trispecific antibody and therefore may be advantageous from a manufacturing standpoint as compared to including an additional antigen binding arm containing a VH, VL and CH1.

The camelidae antigen binding domain (e.g. VHH) may be fused to either the first, second, third or fourth antigen binding arm, typically via a peptide linker. Suitable peptide linkers are known in the art and may consist of 5 to 100 amino acids, 5 to 50 amino acids, 5 to 25 amino acids, or 5 to 15 amino acids. Peptide linkers are formed mainly from glycine and serine amino acid residues and can comprise the amino acid sequences GGGGS or SGGGGS. In one exemplary aspect, the peptide linker comprises or consists of (GGGGS)2.

In some aspects where the third antigen binding arm is fused to the first antigen binding arm, the additional antigen binding domain (e.g. VHH) is fused to the second antigen binding arm. Alternatively, where the third antigen binding arm is fused to the second antigen binding arm, the additional antigen binding domain (e.g. VHH) is fused to the first antigen binding arm.

In some aspects, the additional antigen binding domain (e.g. Fab or VHH) is capable of binding an epitope on CD8. In some aspects, the first antigen binding arm is capable of binding CD3 and the additional antigen binding domain (e.g. VHH) is capable of binding an epitope on CD8.

CD8 (cluster of differentiation 8) is a dimer consisting of a pair of CD8 chains. The most common form of CD8 is composed of a CD8-α and CD8-β chain. CD8 serves as the coreceptor on MHC I-restricted T-cells and acts to enhance the antigen sensitivity of CD8⁺ T-cells by binding to a largely invariant region of MHCI at a site distinct from where the T-cell receptor binds. Without wishing to be bound by theory, including an antigen binding domain (e.g. VHH) that is capable of binding CD8 in the multispecific is believed to allow for preferential activation of CD8⁺ T-cells, which may provide superior therapeutic efficacy.

### Sequence Identity and Mutations

As described herein, a bispecific antibody in the ‘2+1’ format comprises a first, second and third antigen binding arms, where at least one of the antigen binding arms comprises a constant light chain lambda region (CLλ) and a lambda charge pair between the CLλ corresponding CH1. Also described herein are antigen binding arms comprising a constant light chain kappa region (CLκ). Additionally described herein are multispecific antibodies comprising an additional antigen binding region (e.g. VHH), in addition to the binding arms of the ‘2+1’ bispecific antibody.

In some aspects, the CLλ of the light chain or light chains comprises an amino acid sequence having at least 90%, 91%, 92%, 93%, 94%, 95%, 96%, 97%, 98%, or 99% identity to SEQ ID NO: 1 or SEQ ID NO: 2. In some aspects, the CLλ of the first light chain comprises an amino acid sequence of SEQ ID NO: 1 or SEQ ID NO: 2 with 1, 2, 3, 4, 5, 6, 7, 8, 9 or 10 amino acid modifications.

In some aspects, the CLκ of the light chain or light chains (where present) comprises an amino acid sequence having at least 90%, 91%, 92%, 93%, 94%, 95%, 96%, 97%, 98%, or 99% identity to SEQ ID NO: 3. In some aspects, the CLκ (where present) comprises an amino acid sequence of SEQ ID NO: 3 with 1, 2, 3, 4, 5, 6, 7, 8, 9 or 10 amino acid modifications.

In some aspects, the first, second and/or third CH1 comprises an amino acid sequence having at least 90%, 91%, 92%, 93%, 94%, 95%, 96%, 97%, 98%, 99%, or 100% identity to SEQ ID NO: 4 or SEQ ID NO: 5. In some aspects, the CH1 comprises an amino acid sequence of SEQ ID NO: 4 or SEQ ID NO: 5 with 1, 2, 3, 4, 5, 6, 7, 8, 9 or 10 amino acid modifications.

The 1, 2, 3, 4, 5, 6, 7, 8, 9 or 10 amino acid modifications may be in addition to the modifications described above to introduce the charge pairs, engineered disulfides and/or Fc region modifications described above. For example, compared to the wild type CLλ set forth in SEQ ID NO: 1, the CLλ used in the multispecific antibody may contain a lambda charge pair mutation, an engineered disulfide (e.g. S122C and C212V) and 1, 2, 3, 4, 5, 6, 7, 8, 9 or 10 further amino acid modifications. As another example, compared to the wild type CH1 provided in SEQ ID NO: 4, the CH1 used in the multispecific antibody may contain a lambda charge mutation, an engineered disulfide (e.g. F126C, C220V) and 1, 2, 3, 4, 5, 6, 7, 8, 9 or 10 further amino acid modifications.

An amino acid modification may be an insertion, a substitution, or a deletion. In some aspects, the amino acid modification is a substitution of an amino acid residue to any other naturally occurring or non-naturally occurring amino acid residue.

Naturally occurring residues may be divided into classes based on common side chain properties:
1) nonpolar, aliphatic: glycine (G), methionine (M), alanine (A), valine (V), leucine (L), isoleucine (I);
2) polar: cysteine (C), asparagine (N), glutamine (Q), proline (P);
3) polar, partially negatively charged: serine (S), threonine (T);
4) acidic (negatively charged): aspartic acid (D), glutamic acid (E);
5) basic (positively charged): histidine (H), lysine (K), arginine I;
6) aromatic: tryptophan (W), tyrosine (Y), phenylalanine (F).

As described above, serine (S) and threonine (T) have an isoelectric point below 6 and are partially negatively charged at neutral pH, hence they are classed here as ‘polar, partially negatively charged’.

The amino acid substitution may be a conservative amino acid substitution. Conservative amino acid substitutions may involve exchange of a member of one of these classes with another member of the same class. For example, a conservative amino acid substitution may be a substitution of the acidic amino acid glutamic acid (E) for the acidic amino acid aspartic acid (D).

### Nucleic Acids, Vectors and Host Cells

Also provided herein is one or more nucleic acid(s) encoding the multispecific antibody described herein. In some aspects, the nucleic acid(s) is/are purified or isolated, e.g. from other nucleic acid, or naturally-occurring biological material. The skilled person would have no difficulty in preparing such nucleic acid molecules using methods well-known in the art.

In some aspects, the one or more nucleic acids encode a light chain as described herein and/or a CH1 as described herein. The one or more nucleic acid(s) encoding the first or second CH1 may further encode other heavy chain domains, e.g. the hinge, CH2 and CH3, and may encode a complete heavy chain.

The present disclosure also provides one or more vector(s) comprising nucleic acid(s) encoding a multispecific antibody described herein. Suitable vectors can be chosen or constructed, containing appropriate regulatory sequences, including promoter sequences, terminator fragments, polyadenylation sequences, enhancer sequences, marker genes and other sequences as appropriate. In some aspects, the vector contains appropriate regulatory sequences to drive the expression of the nucleic acid in a host cell. Vectors may be plasmids, viral e.g. phage, or phagemid, as appropriate.

The multispecific antibody may be produced from a light chain vector and a heavy chain vector. A light chain vector may contain the nucleic acid encoding the first light chain and the nucleic acid encoding the second light chain acid, which may be present on the vector as separate cassettes (e.g. each operably connected to a different promoter). As described above, the third light chain may be the same as the second light chain and therefore encoded by the same nucleic acid. Alternatively, the third light chain may be a separate nucleic acid.

A heavy chain vector may contain may be used to encode both the first CH1 and first VH (and first Fc region, if present) and second CH1 and second VH (and second Fc region, if present), which may be present on the vector as separate cassettes. As described above, in the ‘2+1’ format, the CH1 of the third antigen binding arm is fused to the VH of either the first antigen binding arm or the second antigen binding arm. Accordingly, if the third antigen binding arm is fused to the first antigen binding arm then the third CH1 and third VH will be encoded by the nucleic acid encoding the first CH1 and first VH, and if the third antigen binding arm is fused to the second antigen binding arm then the third CH1 and third VH will be encoded by the nucleic acid encoding the second CH1 and second VH. The same applies, by extension, to nucleic acids encoding antibodies of the ‘2+2’ format described above. Similarly, where an additional antigen binding domain (e.g. VHH) is present, that is fused to either the VH of either the first antigen binding arm or the second antigen binding arm and therefore will be encoded by the nucleic acid encoding the first CH1 and first VH or the nucleic acid encoding the second CH1 and second VH.

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. In some aspects, the host cell is a mammalian cell, such as a CHO, NS0, or HEK cell, for example a HEK293 cell.

In some aspects, the host cell is a CHO cell.

### Methods of Producing the Multispecific Antibodies

Also provided herein is a method of producing the multispecific antibody described herein.

In some aspects, the method comprises
a) expressing the first, second and third light chain and the first, second and third CH1 in a host cell;
b) allowing the first light chain to pair with the first CH1 so as to form the first binding arm, allowing the second light chain to pair with the second CH1 so as to form the second binding arm, allowing the third light chain to pair with the third CH1, and allowing the first binding arm to pair with the second and third binding arms so as to form the multispecific antibody; and
c) purifying the multispecific antibody from the host cell.

In some aspects, part (a) further comprises expressing the fourth light chain and the fourth CH1 in the host cell. In such cases, part (b) further comprises allowing the fourth light chain to pair with the fourth CH1 to form the fourth binding arm, and allowing the first to fourth binding arms to form pairs so as to form the multispecific antibody.

Expressing the first, second and third, and optionally fourth, light chain and first, second and third, and optionally fourth, CH1 in a host cell may comprise introducing nucleic acids or vectors into host cells (e.g. CHO cells) using suitable techniques as described above. The host cell may then be cultured using suitable techniques, such that the light chain and heavy chain polypeptides pair and form the first and second binding arms. During normal multispecific antibody development, the various light chains and heavy chain polypeptides associate with each other (e.g. through inter-chain disulfide bonds formed between native cysteines, and/or through cysteines engineered into the multispecific antibodies as described herein) and the heavy chains associate with each other (e.g. through inter-chain disulfide bonds formed between cysteines in the two Fc domains). As described herein, the presence of the lambda charge pairs encourages the correct heavy chain/light chain pair to form in the multispecific antibody.

Techniques for the purification of recombinant antibody molecules are well-known in the art and include, for example high performance liquid chromatography, fast protein liquid chromatography, ion exchange chromatography, and affinity chromatography, e.g. using Protein A or Protein L or by binding to an affinity tag. In some aspects, purification is carried out using affinity chromatography (e.g. Protein A affinity chromatography). In some aspects, purification further comprises (e.g. in addition to Protein A chromatography) light chain affinity chromatography. As described herein, light chain affinity chromatography can be used to selectively purify multispecific antibodies containing both CLκ and CLλ and can therefore be used to improve production of multispecific antibodies in this format.

In some aspects, less than 25%, less than 20%, less than 15%, or less than 10%, less than 5%, less than 4%, less than 3%, less than 2%, or less than 1% of the light chains in the multispecific antibodies are mispaired (i.e. paired with a CH1 from a different antigen binding arm) following purification (e.g. by protein A affinity chromatography, or following protein A affinity chromatography and light chain affinity chromatography). Methods for determining the correct light chain pairing are known in the art and include mass spectrometry analysis and microfluidics-based electrophoresis, as described in more detail herein. In some cases the method comprises measuring the correct light chain pairing.

The method may also comprise formulating the antibody molecule into a pharmaceutical composition, optionally with a pharmaceutically acceptable excipient or other substance as described below.

### Treatment

The multispecific antibodies described herein may thus be useful for therapeutic applications, such as in the treatment of cancer.

A multispecific antibody as described herein may be used in a method of treatment of the human or animal body. Related aspects of the disclosure provide;
(i) a multispecific antibody described herein for use as a medicament,
(ii) a multispecific antibody described herein for use in a method of treatment of a disease or disorder,
(iii) a multispecific antibody described herein in the manufacture of a medicament for use in the treatment of a disease or disorder; and,
(iv) a method of treating a disease or disorder in an individual, wherein the method comprises administering to the individual a therapeutically effective amount of a multispecific antibody as described herein.

The individual may be a patient, or more specifically a human patient.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment.

Treatment as a prophylactic measure (i.e. prophylaxis) is also included. For example, an individual susceptible to or at risk of the occurrence or re-occurrence of a disease such as cancer may be treated as described herein. Such treatment may prevent or delay the occurrence or re-occurrence of the disease in the individual.

A method of treatment as described may be comprise administering at least one further treatment to the individual in addition to the multispecific antibody. The multispecific antibody described herein may thus be administered to an individual alone or in combination with one or more other treatments. Where the multispecific antibody is administered to the individual in combination with another treatment, the additional treatment may be administered to the individual concurrently with, sequentially to, or separately from the administration of the multispecific antibody. Where the additional treatment is administered concurrently with the multispecific antibody, the multispecific antibody and additional treatment may be administered to the individual as a combined preparation. For example, the additional therapy may be a known therapy or therapeutic agent for the disease to be treated.

Whilst a multispecific antibody may be administered alone, multispecific antibodies will usually be administered in the form of a pharmaceutical composition, which may comprise at least one component in addition to the multispecific antibody. Another aspect of the disclosure therefore provides a pharmaceutical composition comprising an multispecific antibody as described herein. A method comprising formulating a multispecific antibody into a pharmaceutical composition is also provided.

Pharmaceutical compositions may comprise, in addition to the multispecific antibody, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, and the scheduling of administration.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the disclosure in diverse forms thereof.

While the disclosure has been described in conjunction with the exemplary aspects described above, many equivalent modifications and variations will be apparent to those skilled in the art when given this disclosure. Accordingly, the exemplary aspects of the disclosure set forth above are considered to be illustrative and not limiting. Various changes to the described aspects may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by the use of the antecedent “about,” it will be understood that the particular value forms another aspect. The term “about” in relation to a numerical value is optional and means for example +/−10%.

### EXAMPLES

### Example 1—Design of Charge Pair Variants in Lambda LC-HC Interface

To improve correct chain pairing beyond what alternative disulfides can achieve in DuetMab setting (see WO 2013/096291, incorporated herein by reference), charge pairs were designed using amino acids that participate in lambda light chain (LC)-heavy chain (HC) interface. Pairs of amino acids in lambda LC-HC interface suitable for substitution were identified. The following four criteria were applied: Must be part of the interface.; The Cb atom should be pointing towards the interface (for glycine amino acids carbonyl should be facing in opposite direction from interface); There must be available space beyond Cb to introduce longer amino acids; and there must be at least one allowed combination of Chi angles that places side chain of new amino acids into non-clashing position with existing amino acids.

The following positions were evaluated as lambda light chain amino acids participating in interface formation with a CH1 domain: T117, F119, S122, E124, E125, K130, T132, V134, L136, S138, D139, E161, T163, S166, Q168, A174, S176, Y178, S180, in connection with the following heavy chain CH1 domain amino acids participating in interface formation with a lambda light chain CL domain: S124, F126, L128, A129, S131,S132, K133, S134, A141, G143, L145, K147, D148, H168, F170, P171, V173, Q175, S176, S181, S183, V185, T187, V211, K213.

These amino acids were explored pairwise or alone, one pair at a time or in combinations, with alternative interchain disulfides or keeping disulfides native. Introduction of positively or partially positively charged amino acid means substituting existing amino acids at that position with lysine and arginine and in some cases with asparagine or glutamine or histidine. Introduction of negatively or partially negatively charged amino acid means substituting existing amino acids at that position with aspartic acid, glutamic acid, serine, threonine and in some cases with asparagine or glutamine. Addition of histidine residue at some of these positions will allow to introduce pH dependent CH1-CL interaction.

Nine sets of pair combinations in the lambda LC-HC interface meeting the criteria mentioned above are provided in Table 1 as a non-exhaustive example and were tested for improved pairing.

TABLE 1
All mutations presented here are specific for lambda light chain
containing molecules and expected to perform in wild type as well
as V12 formats (see below). In addition, opposite charge pairs [i.e.,
V134(D, E, S, T)-L128(R, K, H)] are also expected to provide
preferential pairing. Amino acids containing a side chain with no charge
such as asparagine and glutamine can be used for substitutions for
either bearing positive or negative partial charge as they have been
found to participate in formation of hydrogen bonds with both
positively and negatively charged amino acids as well as to each other.

| Set | Cλ (+) | CH1(−) |
|---|---|---|
| #1 | V134 (R, K, H) | L128 (D, E, S, T) |
| #2 | V134 (R, K, H) | L145 (D, E, S, T) |
| #3 | L136 (R, K, H) | V185 (D, E, S, T) |
| #4 | T117 (R, K, H) | V185 (D, E, S, T) |
| #5 | T117 (R, K, H) | A141 (D, E, S, T) |
| #6 | F119 (R, K, H) | L128 (D, E, S, T) |
| #7 | Y178 (R, K, H) | V173 (D, E, S, T) |
| #8 | V134 (R, K, H) | S183 (D, E, T) |
| #9 | T117 (R, K, H) | T187 (D, E) |

### Example 2—Materials and Methods

The materials and methods set forth herein were used to perform the experiments described in subsequent examples. All reagents were from Thermo Fisher Scientific, Waltham, MA, unless stated otherwise. As noted elsewhere, the terms “charge pair(s)” and “charge mutation(s)” are used interchangeably throughout this specification and the amino acid numbering is based on EU numbering system unless specified otherwise.

Construction of pDuet-Heavy and pDuet-Light Mammalian Expression Vectors for DuetMab with Charge Pairs

For construction of DuetMab antibodies with charge pair mutations in heavy chain-light chain interface, the pDuet-Heavy and pDuet-Light plasmids described in (WO 2013/096291 and in Mazor et.al mAbs 2015) were used as backbone vectors. Briefly, the pDuet-Heavy vector contained two human gamma1 heavy chain (HC) cassettes to support HC heterodimerization, where the former heavy chain carried the “Hole” set of mutations (T366S/L368A/Y407V) and a stabilizing mutation (Y349C) in CH3 domain, while the latter carried the complement “Knob” mutation (T366W) and a stabilizing mutation (S354C) in CH3, although the order of the cassettes could readily be reversed. The pDuet-Light vector contained two human light chain (LC) cassettes, where the former light chain carried a kappa constant domain (Cκ), while the latter carried a lambda constant domain (Cλ). The pDuet-Heavy and pDuet-Light vectors also contained the mutations to remove the native interchain disulfide bond in CH1/CA and provide the alternative disulfide bond which is denoted as “V12 DS” or “V12” in this specification, where the mutations F126C/C220V were introduced in the CH1 domain of the “Knob” heavy chain, and mutations S122C/C212V were introduced in the lambda constant domain. The amino acid sequences of the constant domains in the exemplified DuetMab antibody backbones (prior to the introduction of charge mutations) is provided as follows:

| Constant domain in chain: | SEQ ID NO: |
|---|---|
| Hole HC | 13 |
| Kappa LC | 3 |
| Knob V12 HC | 15 |
| Lambda V12 LC | 2 |

The mutations of the “Knob-and-Hole” set and the stabilizing/alternative disulfide bonds utilized herein were provided merely as an example. One skilled in the art can use any other combinations of mutations for “Knob-and-Hole” technique and/or stabilizing/alternative disulfide bonds known in this field to support HC heterodimerization.

For construction of the pDuet-Heavy vector with charge mutations, the “Hole” heavy chain was cloned into the pDuet-Heavy vector by a synthesized DNA fragment of VH-CH1-CH2-CH3 domains containing the above-mentioned mutations for “Hole” heavy chain using restriction cloning technique by BssHII/HindIII. Optionally, the “Hole” heavy chain contained the charge mutation S183K in CH1 domain. The “Knob” heavy chain was cloned into the vector by a synthesized DNA fragment of VH-CH1-CH2-CH3 domains containing the above-mentioned mutations for “Knob” heavy chain using restriction cloning technique by BsrGI/EcoRI. Optionally, the “Knob” heavy chain contained one of the charge mutations in CH1 domain: L128D, L128E, L128S, L128T, A141D, A141E, A141S, A141T, L145D, L145E, L145S, L145T, S183D, V185D, V185E, V185S, V185T, V173D, V173E, V173S, and V173T.

For construction of the pDuet-Light with charge mutations, the kappa light chain was cloned into the pDuet-Light vector by a synthesized DNA fragment of VL-Cκ domains using restriction cloning technique by BssHII/NheI. Optionally, the constant kappa (Cκ) domain contained the charge mutation V133E. The lambda light chain was cloned into the pDuet-Light vector by a synthesized DNA fragment of VL-Cλ domains containing the above-mentioned S122C/C212V mutations for lambda light chain using restriction cloning technique by BsrGI/EcoRI. Optionally, the constant lambda (Cλ) domain contained one of the charge mutations: V117R, V117K, F119R, F119K, V134R, V134K, L136R, L136K, Y178R, and Y178K. The light chain variable domain (VL) could be either variable kappa domain (Vκ) or variable lambda domain (Vλ).

### Expression, Affinity Purification and Protein Quantification

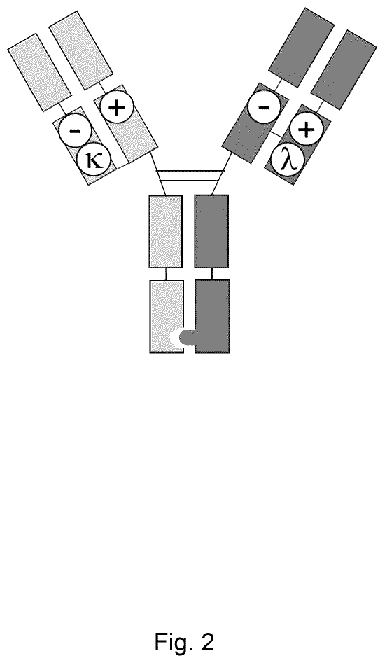

All constructs were transiently expressed in CHO cells in suspension using PEI-MAX (Polysciences, Inc., Warrington, PA) as a transfection reagent and grown in an in-house made CHO medium. The vectors containing the following combinations of charge pairs were used for the expression of the antibodies in these studies. A schematic of the constructed DuetMabs containing charge pairs is provided in FIG. 2. The bispecific antibodies were generated against several different antigens expressed on the surface of cells, referred to here as Antigen 1, 2, 3, 4, 5 and 6. Antigen 3 is CD3. The generated bispecific antibodies were referred as “Target1/Target2-DuetMab” or simply “Target1/Target2”:

TABLE 2
list of charge pair variants generated.

| | pDuet-Heavy | | | | pDuet-Light | | | |
|---|---|---|---|---|---|---|---|---|
| Sample | Hole Heavy Chain | | Knob Heavy Chain | | Kappa Light Chain | | Lambda Light Chain | |
| # | Target1 | CH1 | Target2 | CH1 (V12) | Target1 | CL | Target2 | CL (V12) |
| 1 | Antigen 1 | WT | Antigen 2 | WT | Antigen 1 | WT | Antigen 2 | WT |
| 2 | Antigen 1 | S183K | Antigen 2 | WT | Antigen 1 | V133E | Antigen 2 | N/A |
| 3 | Antigen 1 | S183K | Antigen 2 | L128D | Antigen 1 | V133E | Antigen 2 | V134R |
| 4 | Antigen 1 | S183K | Antigen 2 | L128E | Antigen 1 | V133E | Antigen 2 | V134R |
| 5 | Antigen 1 | S183K | Antigen 2 | L128S | Antigen 1 | V133E | Antigen 2 | V134R |
| 6 | Antigen 1 | S183K | Antigen 2 | L128T | Antigen 1 | V133E | Antigen 2 | V134R |
| 7 | Antigen 1 | S183K | Antigen 2 | L145D | Antigen 1 | V133E | Antigen 2 | V134R |
| 8 | Antigen 1 | S183K | Antigen 2 | L145E | Antigen 1 | V133E | Antigen 2 | V134R |
| 9 | Antigen 1 | S183K | Antigen 2 | L145S | Antigen 1 | V133E | Antigen 2 | V134R |
| 10 | Antigen 1 | S183K | Antigen 2 | L145T | Antigen 1 | V133E | Antigen 2 | V134R |
| 11 | Antigen 1 | S183K | Antigen 2 | L128D | Antigen 1 | V133E | Antigen 2 | V134K |
| 12 | Antigen 1 | S183K | Antigen 2 | L128E | Antigen 1 | V133E | Antigen 2 | V134K |
| 13 | Antigen 1 | S183K | Antigen 2 | L128S | Antigen 1 | V133E | Antigen 2 | V134K |
| 14 | Antigen 1 | S183K | Antigen 2 | L128T | Antigen 1 | V133E | Antigen 2 | V134K |
| 15 | Antigen 1 | S183K | Antigen 2 | L145D | Antigen 1 | V133E | Antigen 2 | V134K |
| 16 | Antigen 1 | S183K | Antigen 2 | L145E | Antigen 1 | V133E | Antigen 2 | V134K |
| 17 | Antigen 1 | S183K | Antigen 2 | L145S | Antigen 1 | V133E | Antigen 2 | V134K |
| 18 | Antigen 1 | S183K | Antigen 2 | L145T | Antigen 1 | V133E | Antigen 2 | V134K |
| 19 | Antigen 1 | S183K | Antigen 2 | WT | Antigen 1 | V133E | Antigen 2 | V134K |
| 20 | Antigen 1 | S183K | Antigen 2 | S183D | Antigen 1 | V133E | Antigen 2 | V134K |
| 21 | Antigen 1 | S183K | Antigen 2 | V185D | Antigen 1 | V133E | Antigen 2 | L136R |
| 22 | Antigen 1 | S183K | Antigen 2 | V185E | Antigen 1 | V133E | Antigen 2 | L136R |
| 23 | Antigen 1 | S183K | Antigen 2 | V185S | Antigen 1 | V133E | Antigen 2 | L136R |
| 24 | Antigen 1 | S183K | Antigen 2 | V185T | Antigen 1 | V133E | Antigen 2 | L136R |
| 25 | Antigen 1 | S183K | Antigen 2 | V185D | Antigen 1 | V133E | Antigen 2 | L136K |
| 26 | Antigen 1 | S183K | Antigen 2 | V185E | Antigen 1 | V133E | Antigen 2 | L136K |
| 27 | Antigen 1 | S183K | Antigen 2 | V185S | Antigen 1 | V133E | Antigen 2 | L136K |
| 28 | Antigen 1 | S183K | Antigen 2 | V185T | Antigen 1 | V133E | Antigen 2 | L136K |
| 29 | Antigen 1 | S183K | Antigen 2 | V185D | Antigen 1 | V133E | Antigen 2 | T117R |
| 30 | Antigen 1 | S183K | Antigen 2 | V185E | Antigen 1 | V133E | Antigen 2 | T117R |
| 31 | Antigen 1 | S183K | Antigen 2 | V185S | Antigen 1 | V133E | Antigen 2 | T117R |
| 32 | Antigen 1 | S183K | Antigen 2 | V185T | Antigen 1 | V133E | Antigen 2 | T117R |
| 33 | Antigen 1 | S183K | Antigen 2 | A141D | Antigen 1 | V133E | Antigen 2 | T117R |
| 34 | Antigen 1 | S183K | Antigen 2 | A141E | Antigen 1 | V133E | Antigen 2 | T117R |
| 35 | Antigen 1 | S183K | Antigen 2 | A141S | Antigen 1 | V133E | Antigen 2 | T117R |
| 36 | Antigen 1 | S183K | Antigen 2 | A141T | Antigen 1 | V133E | Antigen 2 | T117R |
| 37 | Antigen 1 | S183K | Antigen 2 | V185D | Antigen 1 | V133E | Antigen 2 | T117K |
| 38 | Antigen 1 | S183K | Antigen 2 | V185E | Antigen 1 | V133E | Antigen 2 | T117K |
| 39 | Antigen 1 | S183K | Antigen 2 | V185S | Antigen 1 | V133E | Antigen 2 | T117K |
| 40 | Antigen 1 | S183K | Antigen 2 | V185T | Antigen 1 | V133E | Antigen 2 | T117K |
| 41 | Antigen 1 | S183K | Antigen 2 | A141D | Antigen 1 | V133E | Antigen 2 | T117K |
| 42 | Antigen 1 | S183K | Antigen 2 | A141E | Antigen 1 | V133E | Antigen 2 | T117K |
| 43 | Antigen 1 | S183K | Antigen 2 | A141S | Antigen 1 | V133E | Antigen 2 | T117K |
| 44 | Antigen 1 | S183K | Antigen 2 | A141T | Antigen 1 | V133E | Antigen 2 | T117K |
| 45 | Antigen 1 | S183K | Antigen 2 | L128D | Antigen 1 | V133E | Antigen 2 | F119R |
| 46 | Antigen 1 | S183K | Antigen 2 | L128E | Antigen 1 | V133E | Antigen 2 | F119R |
| 47 | Antigen 1 | S183K | Antigen 2 | L128S | Antigen 1 | V133E | Antigen 2 | F119R |
| 48 | Antigen 1 | S183K | Antigen 2 | L128T | Antigen 1 | V133E | Antigen 2 | F119R |
| 49 | Antigen 1 | S183K | Antigen 2 | L128D | Antigen 1 | V133E | Antigen 2 | F119K |
| 50 | Antigen 1 | S183K | Antigen 2 | L128E | Antigen 1 | V133E | Antigen 2 | F119K |
| 51 | Antigen 1 | S183K | Antigen 2 | L128S | Antigen 1 | V133E | Antigen 2 | F119K |
| 52 | Antigen 1 | S183K | Antigen 2 | L128T | Antigen 1 | V133E | Antigen 2 | F119K |
| 53 | Antigen 1 | S183K | Antigen 2 | V173D | Antigen 1 | V133E | Antigen 2 | Y178R |
| 54 | Antigen 1 | S183K | Antigen 2 | V173E | Antigen 1 | V133E | Antigen 2 | Y178R |
| 55 | Antigen 1 | S183K | Antigen 2 | V173S | Antigen 1 | V133E | Antigen 2 | Y178R |
| 56 | Antigen 1 | S183K | Antigen 2 | V173T | Antigen 1 | V133E | Antigen 2 | Y178R |
| 57 | Antigen 1 | S183K | Antigen 2 | V173D | Antigen 1 | V133E | Antigen 2 | Y178K |
| 58 | Antigen 1 | S183K | Antigen 2 | V173E | Antigen 1 | V133E | Antigen 2 | Y178K |
| 59 | Antigen 1 | S183K | Antigen 2 | V173S | Antigen 1 | V133E | Antigen 2 | Y178K |
| 60 | Antigen 1 | S183K | Antigen 2 | V173T | Antigen 1 | V133E | Antigen 2 | Y178K |
| 1 | Antigen 1 | WT | Antigen 3 | WT | Antigen 1 | WT | Antigen 3 | WT |
| 2 | Antigen 1 | S183K | Antigen 3 | WT | Antigen 1 | V133E | Antigen 3 | WT |
| 33 | Antigen 1 | S183K | Antigen 3 | A141D | Antigen 1 | V133E | Antigen 3 | T117R |
| 34 | Antigen 1 | S183K | Antigen 3 | A141E | Antigen 1 | V133E | Antigen 3 | T117R |
| 35 | Antigen 1 | S183K | Antigen 3 | A141S | Antigen 1 | V133E | Antigen 3 | T117R |
| 36 | Antigen 1 | S183K | Antigen 3 | A141T | Antigen 1 | V133E | Antigen 3 | T117R |
| 41 | Antigen 1 | S183K | Antigen 3 | A141D | Antigen 1 | V133E | Antigen 3 | T117K |
| 1 | Antigen 4 | WT | Isotype control | WT | Antigen 4 | WT | Isotype control | WT |
| 2 | Antigen 4 | S183K | Isotype control | WT | Antigen 4 | V133E | Isotype control | WT |
| 33 | Antigen 4 | S183K | Isotype control | A141D | Antigen 4 | V133E | Isotype control | T117R |
| 34 | Antigen 4 | S183K | Isotype control | A141E | Antigen 4 | V133E | Isotype control | T117R |
| 35 | Antigen 4 | S183K | Isotype control | A141S | Antigen 4 | V133E | Isotype control | T117R |
| 36 | Antigen 4 | S183K | Isotype control | A141T | Antigen 4 | V133E | Isotype control | T117R |
| 41 | Antigen 4 | S183K | Isotype control | A141D | Antigen 4 | V133E | Isotype control | T117K |
| 1 | Antigen 5 | WT | Antigen 3 | WT | Antigen 5 | WT | Antigen 3 | WT |
| 2 | Antigen 5 | S183K | Antigen 3 | WT | Antigen 5 | V133E | Antigen 3 | WT |
| 33 | Antigen 5 | S183K | Antigen 3 | A141D | Antigen 5 | V133E | Antigen 3 | T117R |
| 34 | Antigen 5 | S183K | Antigen 3 | A141E | Antigen 5 | V133E | Antigen 3 | T117R |
| 35 | Antigen 5 | S183K | Antigen 3 | A141S | Antigen 5 | V133E | Antigen 3 | T117R |
| 36 | Antigen 5 | S183K | Antigen 3 | A141T | Antigen 5 | V133E | Antigen 3 | T117R |
| 41 | Antigen 5 | S183K | Antigen 3 | A141D | Antigen 5 | V133E | Antigen 3 | T117K |
| 1 | Antigen 6 | WT | Antigen 1 | WT | Antigen 6 | WT | Antigen 1 | WT |
| 2 | Antigen 6 | S183K | Antigen 1 | WT | Antigen 6 | V133E | Antigen 1 | WT |
| 33 | Antigen 6 | S183K | Antigen 1 | A141D | Antigen 6 | V133E | Antigen 1 | T117R |
| 34 | Antigen 6 | S183K | Antigen 1 | A141E | Antigen 6 | V133E | Antigen 1 | T117R |
| 35 | Antigen 6 | S183K | Antigen 1 | A141S | Antigen 6 | V133E | Antigen 1 | T117R |
| 36 | Antigen 6 | S183K | Antigen 1 | A141T | Antigen 6 | V133E | Antigen 1 | T117R |
| 41 | Antigen 6 | S183K | Antigen 1 | A141D | Antigen 6 | V133E | Antigen 1 | T117K |

The culture medium was collected 7 to 13 days after transfection and filtered through a 0.22 μm sterile filter. Antibody concentration in culture supernatants was measured by an Octet384 instrument using protein A sensors (Sartorius, Gottingen, Germany) according to the manufacturer's protocol. Antibodies were purified by either protein A magnetic bead affinity purification (Genscript, Piscataway, NJ) or standard protein A affinity chromatography (Cytiva, Marlborough, MA), followed by light chain affinity chromatography if necessary, in accordance with the manufacturer's protocol, and were subsequently buffer exchanged in PBS (pH 7.2). The purity and oligomeric state of purified molecules was determined by microfluidics-based electrophoresis and analytical size exclusion chromatography (see methods below). Protein aggregates were removed by preparative SEC. The concentrations of the purified antibodies were determined by reading the absorbance at 280 nm using theoretically determined extinction coefficients.

### Size-Exclusion Chromatography (SEC)

Analytical SEC-HPLC (Agilent 1260 Infinity HPLC system) was performed using a TSK-gel G3000SWxL column (Tosoh Biosciences, King of Prussia, PA) to determine the oligomeric state of purified molecules. Preparative SEC-HPLC was carried out using a Superdex 200 column (Cytiva) to remove protein aggregates.

### Microfluidics-Based Electrophoresis

Microfluidics-based electrophoresis was performed using Bioanalyzer in accordance with the manufacturer's protocol (Agilent, Santa Clara, CA), in order to assess the ratio of kappa and lambda light chains of an antibody, based on which the percentage of correct light chain ratio was calculated.

### Binding Kinetics Assay

Binding kinetics were measured by biolayer interferometry on an Octet384 instrument. Streptavidin (SA) biosensors were loaded with biotinylated protein antigens (ACRO Biosystems, Newark, DE) in PBS pH 7.2, 1 mg/ml BSA, 0.05% (v/v) TWEEN (Kinetic buffer). The loaded biosensors were washed in the same buffer before carrying out association and dissociation measurements with various antibodies for the indicated times. Kinetic parameters (Kₒₙ and K_{off}) and affinities (K_{D}) were calculated from a non-linear fit of the data using the Octet384 software v.12.2.1.24.

### Accelerated Stability Study

Protein test samples were diluted to 1 mg/mL in PBS (pH 7.2) and split into 3 equal aliquots to serve as control, heat, and photo stress samples. Control samples were incubated at 4° C. for 14 days, heat stress samples were incubated at 45° C. for 14 days, and photo stress samples were incubated in glass vials in an ICH compliant photostability chamber exposed to 3000 lux cool white light for 7 days at 25° C. Samples were then analyzed by HP-SEC to determine levels of aggregate, monomer, and fragment.

### Differential Scanning Fluorimetry (DSF)

Samples were prepared by combining 20 μL of protein sample at 1 mg/mL in PBS (pH 7.2) with 5 μL of SYPRO Orange dye diluted to 40× in PBS (pH 7.2) in a 96-well PCR plate in duplicate. The plate was sealed, and measurements performed in a QuantStudio 7 Flex Real-Time PCR System. Samples were subjected to an initial equilibration step at 25° C. for 2 minutes, followed by a temperature ramp to 99° C. at 0.05° C./sec increments. The fluorescence emission was monitored using the FAM filter set. The Tm value for each sample was calculated in the Protein Thermal Shift™ software using the Boltzmann method.

### Subunit LC-MS Analysis

Subunit LC/MS analysis was performed to characterize the mis-paired species. 50 μg of sample was dried and further reconstituted in 50 μL of 100 mM sodium phosphate buffer, pH 7.0. Digestion was performed by adding 60 units of FabALACTICA enzyme (IgdE) (Genovis AB, Lund, Sweden) to each sample and incubating at 37° C. for 16-18 hours. Waters ACQUITY UPLC system (Waters, Milford, MA) coupled with Waters Xevo G2-XS QTof mass spectrometer were used for subunits separation and mass determination. Two μg of digested subunits were injected in Waters BioResolve RP mAb polyphenyl column (2.1×150 mm, 2.7 mm, 450 Å) for separation. Mobile phase A contained 0.1% formic acid (FA), 0.01% trifluoroacetic acid (TFA) in water, and mobile phase B contained 0.1% FA, 0.01% TFA in water in ACN. A gradient of 25% B to 45% B was performed for 40 minutes at a flow rate of 0.2 mL/min. Column temperature was set at 75° C. The UV profile of eluted subunits were acquired at a wavelength of 280 nm.

### Differential Scanning Calorimetry Analysis (DSC)

DSC experiments were carried out using a MICROCAL VP-DSC scanning microcalorimeter (Malvern, Northampton, MA). Prior to DSC analysis, all samples were diluted to ˜0.6 mg/mL in phosphate buffer saline (PBS, pH 7.2). Exact concentrations were determined from duplicate measurements using a UV-VIS spectrophotometer (NanoDrop 2000C). 400 μL of each sample and corresponding buffer (PBS, pH 7.2) were loaded into a 96-well plate and stored at 10° C. in the autosampler chamber until analysis. All DSC measurements used a temperature window from 20° C. to 100° C. at a scan rate of 60° C./hr. Prior to sample measurement, baseline measurements (buffer-versus-buffer) were obtained for subtraction from the sample measurement. Data analysis, baseline correction, and deconvolution were carried out using the Origin™ DSC software provided by Microcal. Baseline correction was performed using the Linear Connect function within the software. Deconvolution analysis was performed using a non-two-state model and best fits were obtained using 1 and 200-iteration cycles until the chi-square value was minimized. The interpretation of the DSC deconvolution results was based on the fact that the different domains in the antibody formats unfold independently. The Tₒₙₛₑₜ value is defined as the temperature at which the thermogram begins to significantly increase from the baseline. The Tₘ value is defined as the temperature value corresponding to each peak maximum on the thermogram or the deconvoluted thermogram.

### Cell Viability Assay

Cell viabilities were determined using CellTiter-Glo™ Luminescent Cell Viability Assay (Promega). This assay quantifies the ATP present, which signals the presence of metabolically active cells. Luminescence, produced by the luciferase-catalyzed reaction of luciferin and ATP, was measured using a luminescent plate reader. In brief, target cells expressing Antigen 1 were seeded in 96-well plates at a density of ˜1×10⁴ cells/well in RPMI 1640 media supplemented with 0.1% BSA, and 0.2 ng/ml human recombinant EGF. Antibodies at various concentrations were added to triplicate samples, and the cells were incubated for 72 hours at 37° C. and 5% C02 in a humidified incubator. After treatment, the cells were exposed to CellTiter-Glo® reagent (Promega) for ˜ 15 min and OD409 was measured using an EnVision 2104 Multilabel plate reader (PerkinElmer). Cell viability was determined by measuring the ATP level relative to a no-antibody control.

### Example 3—Expression and Properties of Charge Pair Variants

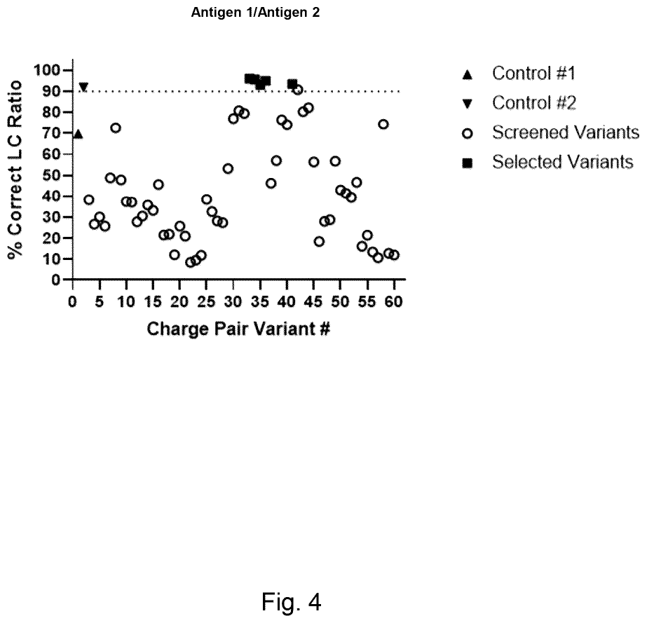

Table 3 and FIG. 4 summarize the expression and biochemical profiles of Antigen 1/Antigen 2 DuetMabs carrying the proposed sets of charge pairs produced in small scale of cell culture (3 mL). FIG. 4 shows the correct LC ratio data of Table 1 plotted in scatter X-Y chart. Compared to controls #1 and #2, charge pair variants #33, #34, #35, #36, and #41 demonstrated improved correct LC ratio and were selected for additional analysis.

TABLE 3
Summary of the expression and biochemical profiles of Antigen 1/Antigen 2 DuetMabs carrying
the proposed sets of charge pairs produced in small scale of cell culture (3 mL). The
antibodies were purified by protein A magnetic bead affinity purification.

| Residue | | Antigen 1 (Hole arm) | | Antigen 2 (Knob arm) | | Day 7 Titer | % Correct |
|---|---|---|---|---|---|---|---|
| Set | Sample # | CH1 | Cκ | CH1 (V12) | Cλ (V12) | (μg/mL) | LC ratio |
| Control | 1 | WT | WT | WT | WT | 183.5 | 69.8 |
| | 2 | S183K | V133E | WT | WT | 121.9 | 91.8 |
| A | 3 | S183K | V133E | L128D | V134R | 93.7 | 38.4 |
| | 4 | S183K | V133E | L128E | V134R | 125.0 | 26.7 |
| | 5 | S183K | V133E | L128S | V134R | 130.3 | 30.1 |
| | 6 | S183K | V133E | L128T | V134R | 104.6 | 25.8 |
| B | 7 | S183K | V133E | L145D | V134R | 166.3 | 48.8 |
| | 8 | S183K | V133E | L145E | V134R | 157.8 | 72.6 |
| | 9 | S183K | V133E | L145S | V134R | 163.2 | 47.8 |
| | 10 | S183K | V133E | L145T | V134R | 129.0 | 37.5 |
| A | 11 | S183K | V133E | L128D | V134K | 127.8 | 37.2 |
| | 12 | S183K | V133E | L128E | V134K | 108.2 | 27.9 |
| | 13 | S183K | V133E | L128S | V134K | 137.7 | 30.6 |
| | 14 | S183K | V133E | L128T | V134K | 149.2 | 35.8 |
| B | 15 | S183K | V133E | L145D | V134K | 158.1 | 33.3 |
| | 16 | S183K | V133E | L145E | V134K | 155.9 | 45.6 |
| | 17 | S183K | V133E | L145S | V134K | 193.8 | 21.6 |
| | 18 | S183K | V133E | L145T | V134K | 130.5 | 21.9 |
| H | 19 | S183K | V133E | WT | V134K | 158.7 | 12.2 |
| | 20 | S183K | V133E | S183D | V134K | 130.9 | 25.8 |
| C | 21 | S183K | V133E | V185D | L136R | 169.0 | 21.0 |
| | 22 | S183K | V133E | V185E | L136R | 167.0 | 8.6 |
| | 23 | S183K | V133E | V185S | L136R | 138.3 | 9.7 |
| | 24 | S183K | V133E | V185T | L136R | 148.8 | 11.9 |
| | 25 | S183K | V133E | V185D | L136K | 128.5 | 38.5 |
| | 26 | S183K | V133E | V185E | L136K | 129.3 | 32.7 |
| | 27 | S183K | V133E | V185S | L136K | 151.1 | 28.2 |
| | 28 | S183K | V133E | V185T | L136K | 143.5 | 27.4 |
| D | 29 | S183K | V133E | V185D | T117R | 130.4 | 53.2 |
| | 30 | S183K | V133E | V185E | T117R | 137.2 | 77.0 |
| | 31 | S183K | V133E | V185S | T117R | 150.9 | 80.8 |
| | 32 | S183K | V133E | V185T | T117R | 144.4 | 79.5 |
| E | 33 | S183K | V133E | A141D | T117R | 242.2 | 96.0 |
| | 34 | S183K | V133E | A141E | T117R | 256.8 | 95.6 |
| | 35 | S183K | V133E | A141S | T117R | 214.0 | 93.1 |
| | 36 | S183K | V133E | A141T | T117R | 132.4 | 95.0 |
| D | 37 | S183K | V133E | V185D | T117K | 89.0 | 46.2 |
| | 38 | S183K | V133E | V185E | T117K | 108.3 | 57.0 |
| | 39 | S183K | V133E | V185S | T117K | 152.0 | 76.4 |
| | 40 | S183K | V133E | V185T | T117K | 131.7 | 74.1 |
| E | 41 | S183K | V133E | A141D | T117K | 167.7 | 93.5 |
| | 42 | S183K | V133E | A141E | T117K | 177.0 | 90.8 |
| | 43 | S183K | V133E | A141S | T117K | 167.0 | 80.4 |
| | 44 | S183K | V133E | A141T | T117K | 119.9 | 82.2 |
| F | 45 | S183K | V133E | L128D | F119R | 79.1 | 56.4 |
| | 46 | S183K | V133E | L128E | F119R | 99.5 | 18.5 |
| | 47 | S183K | V133E | L128S | F119R | 100.3 | 28.0 |
| | 48 | S183K | V133E | L128T | F119R | 87.4 | 28.8 |
| | 49 | S183K | V133E | L128D | F119K | 48.2 | 56.7 |
| | 50 | S183K | V133E | L128E | F119K | 106.0 | 43.0 |
| | 51 | S183K | V133E | L128S | F119K | 91.7 | 41.4 |
| | 52 | S183K | V133E | L128T | F119K | 67.4 | 39.5 |
| G | 53 | S183K | V133E | V173D | Y178R | 95.3 | 46.7 |
| | 54 | S183K | V133E | V173E | Y178R | 89.6 | 16.2 |
| | 55 | S183K | V133E | V173S | Y178R | 105.7 | 21.5 |
| | 56 | S183K | V133E | V173T | Y178R | 94.6 | 13.5 |
| | 57 | S183K | V133E | V173D | Y178K | 139.1 | 10.8 |
| | 58 | S183K | V133E | V173E | Y178K | 94.0 | 74.3 |
| | 59 | S183K | V133E | V173S | Y178K | 130.0 | 12.9 |
| | 60 | S183K | V133E | V173T | Y178K | 105.3 | 12.1 |

Table 4 summarizes the expression (Table 4A) and biochemical profiles (Table 4B) of Antigen 1/Antigen 2 DuetMabs carrying the selected charge pair variants #1, #2, #33, #34, #35, #36, and #41 produced in large scale of cell culture (100 mL). The biochemical profiles of the selected DuetMabs were consistent despite of the production scale. For additional analysis, the DuetMabs were further purified by light chain affinity chromatography to remove mispaired byproducts, and aggregates were removed by preparative SEC.

TABLE 4A
Summary of expression data for selected charge pair samples #1, #2, #33,
#34, #35, #36, and #41 produced in large scale of cell culture (100 mL).

| | Antigen 1 (Hole arm) | | Antigen 2 (Knob arm) | | Titer (ug/mL) | | |
|---|---|---|---|---|---|---|---|
| Sample # | CH1 | Cκ | CH1 (V12) | Cλ (V12) | Day 7 | Day 10 | Day 14 |
| 1 | WT | WT | WT | WT | 99.8 | 228.4 | 361 |
| 2 | S183K | V133E | WT | WT | 83.5 | 189.2 | 316.8 |
| 33 | S183K | V133E | A141D | T117R | 86.6 | 190.2 | 291 |
| 34 | S183K | V133E | A141E | T117R | 99.6 | 218.3 | 342.9 |
| 35 | S183K | V133E | A141S | T117R | 107.7 | 237 | 383 |
| 36 | S183K | V133E | A141T | T117R | 115.4 | 247.1 | 388.5 |
| 41 | S183K | V133E | A141D | T117K | 40.4 | 82.2 | 103.9 |

TABLE 4B
Summary of biochemical profiles for selected charge pair samples #1, #2, #33, #34, #35, #36,
and #41 produced in large scale of cell culture (100 mL). For further biochemical, biophysical and
biological profiling, antibodies were purified by protein A affinity chromatography followed by light chain
affinity chromatography, and then subjected to preparative SEC for aggregate removal.

| | | | Antigen 2 (Knob | | Profiles after protein A | | Profiles after Light Chain | |
|---|---|---|---|---|---|---|---|---|
| | Antigen 1 (Hole | | arm) | | purification | | Affinity Purification | |
| | arm) | | CH1 | Cλ | % Correct | % | % Correct | % |
| Sample # | CH1 | C_{K} | (V12) | (V12) | LC ratio | Monomer | LC ratio | Monomer |
| 1 | WT | WT | WT | WT | 64.9 | 91.9 | 90.9 | >99 |
| 2 | S183K | V133E | WT | WT | 92.1 | 91.7 | 93.6 | >99 |
| 33 | S183K | V133E | A141D | T117R | 98.6 | 93.5 | 99.7 | >99 |
| 34 | S183K | V133E | A141E | T117R | 97.1 | 92.3 | 99.7 | >99 |
| 35 | S183K | V133E | A141S | T117R | 90.2 | 94.3 | 95.3 | >99 |
| 36 | S183K | V133E | A141T | T117R | 99.7 | 92.5 | 93.8 | >99 |
| 41 | S183K | V133E | \|A141D | T117K | 96.3 | 92.5 | 99.0 | >99 |

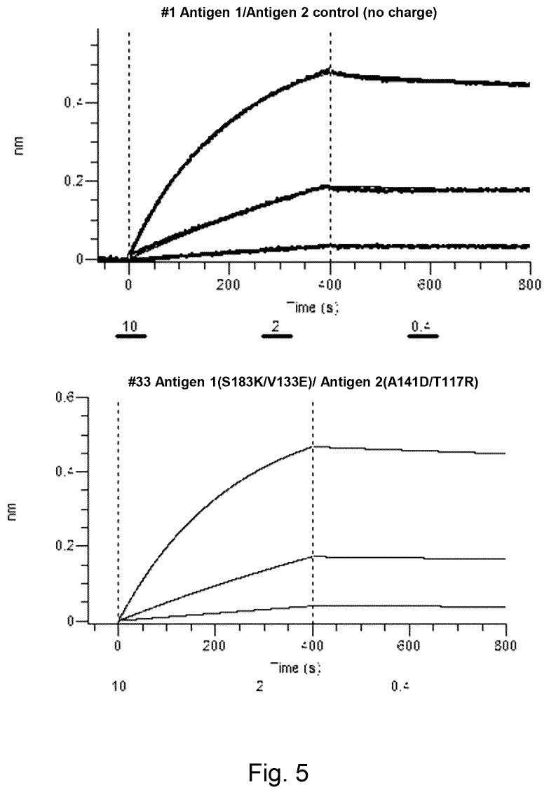

FIG. 5 and Table 5 show binding kinetics of Antigen 1/Antigen 2 DuetMabs carrying the selected charge pair variants. FIG. 5 shows the response signals and fitting curves of control sample #1 and variant #33, which is representative of the tested variants. The binding affinities of variants #33, #34, #35, #36, and #41 for Antigen 2 were comparable to controls #1 and #2.

TABLE 5
Kinetics measurements to soluble monomeric form of Antigen 2 were obtained using an Ocet384 instrument.
The dissociation constants, K_{D}, were calculated as a ratio of k_{off}/kₒₙ from a non-linear fit of the data.

| Sample | Antigen 1 | Antigen 2 | | | | | kdis | kdis | Full | Full |
|---|---|---|---|---|---|---|---|---|---|---|
| # | (CH1/Cκ) | (CH1/Cλ) | KD (M) | KD Error | ka (1/Ms) | ka Error | (1/s) | Error | X\hat{ }2 | R\hat{ }2 |
| 1 | WT | WT | 3.45E−10 | 2.58E−12 | 5.20E+05 | 9.11E+02 | 1.80E−04 | 1.30E−06 | 0.2098 | 0.9994 |
| 2 | S183K/V133E | WT | 5.08E−10 | 1.83E−12 | 4.89E+05 | 5.64E+02 | 2.48E−04 | 8.45E−07 | 0.0905 | 0.9998 |
| 33 | S183K/V133E | A141D/T117R | 2.43E−10 | 2.11E−12 | 4.19E+05 | 5.69E+02 | 1.02E−04 | 8.74E−07 | 0.0893 | 0.9997 |
| 34 | S183K/V133E | A141E/T117R | 4.13E−10 | 2.17E−12 | 5.67E+05 | 8.45E+02 | 2.34E−04 | 1.18E−06 | 0.1941 | 0.9995 |
| 35 | S183K/V133E | A141S/T117R | 4.72E−10 | 1.79E−12 | 4.43E+05 | 4.90E+02 | 2.09E−04 | 7.59E−07 | 0.0821 | 0.9998 |
| 36 | S183K/V133E | A141T/T117R | 4.04E−10 | 1.97E−12 | 4.86E+05 | 6.21E+02 | 1.96E−04 | 9.23E−07 | 0.1196 | 0.9997 |
| 41 | S183K/V133E | A141D/T117K | 4.90E−10 | 1.37E−12 | 5.03E+05 | 4.45E+02 | 2.46E−04 | 6.53E−07 | 0.0567 | 0.9999 |

Table 6 summarizes the thermal stabilities of Antigen 1/Antigen 2 DuetMabs carrying the selected charge pair variants by differential scanning fluorimetry (DSF) and their accelerated stability profiles. NIP228 served as an IgG1 control. The Antigen 1/Antigen 2 DuetMab variants showed no concerns for aggregation or fragmentation after heat stress. HP-SEC retention times of the Antigen 1/Antigen 2 DuetMab variants are consistent with that of NIP228 IgG1 control (ART from NIP228<0.2m). DSF values showed no significant difference among the charge pair variants and were consistent with that of NIP228 IgG1 control.

TABLE 6
Accelerated stability measurements of variants were measured after a 14-day incubation
at 45° C. by SEC. DSF values recorded the Tₒₙₛₑₜ and Tₘ of the selected Variants.

| | Accelerated Stability (45° C., 14 d) | | | | | |
|---|---|---|---|---|---|---|
| | Monomer | Aggregate | Fragment | ΔRT from | DSF | |
| Sample | Loss (<5%) | Increase (<1%) | Increase (<4%) | NIP228 (<0.2 m) | Tₒₙₛₑₜ | Tₘ |
| Control #1 | −1.20 | 0.10 | 1.10 | 0.16 | 56.91 | 66.44 |
| Control #2 | −1.10 | 0.00 | 1.10 | 0.15 | 56.74 | 66.16 |
| Variant #33 | −1.90 | 0.00 | 1.90 | 0.14 | 57.09 | 66.41 |
| Variant #34 | −1.13 | 0.00 | 1.13 | 0.14 | 56.74 | 65.91 |
| Variant #35 | −3.67 | 0.00 | 3.67 | 0.15 | 57.55 | 66.04 |
| Variant #36 | −1.15 | 0.00 | 1.15 | 0.15 | 57.61 | 66.14 |
| Variant #41 | −1.10 | 0.00 | 1.10 | 0.14 | 57.79 | 66.28 |

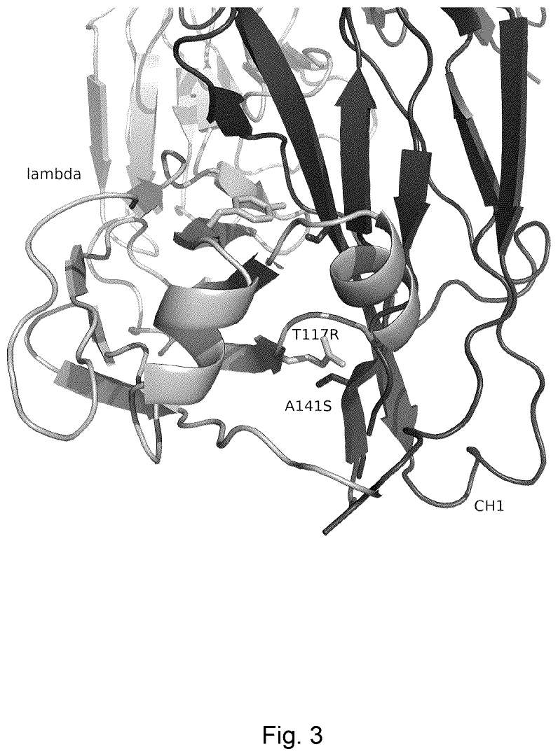

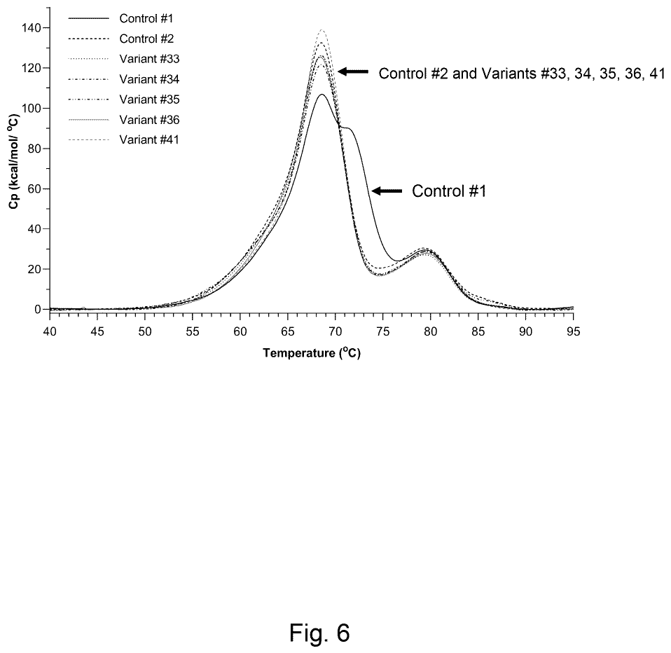

FIG. 6 and Table 7 show the thermal stability studies of Antigen 1/Antigen 2 DuetMabs carrying the selected charge pair variants using differential scanning calorimetry (DSC) analysis. FIG. 3 illustrates the stacked thermograms for Antigen 1/Antigen 2 DuetMab variants. Deconvolution of the thermograms revealed the transitions for the Fab, CH2, and CH3 domains, where some transitions were overlapped and under the same TM peaks. Table 7 lists the deconvoluted TM and approximated Tₒₙₛₑₜ values of Antigen 1/Antigen 2 DuetMab charge pair variants. All the variants had similar approximated Tₒₙₛₑₜ values, indicating the selected charge pairs did not significantly impact thermostability.

TABLE 7
DSC thermostability measurements captured transitions for the Fab,
CH2, and CH3 domains under the T_{M1}, T_{M2}, T_{M3} and T_{M4} descriptions. Some
transitions are under the same T_{M} peak transition.

| | | | | | | | Approximated |
|---|---|---|---|---|---|---|---|
| | Antigen 1 | Antigen 2 | T_{M1} | T_{M2} | T_{M3} | T_{M4} | Tₒₙₛₑₜ |
| Sample | (CH1/Cκ) | (CH1/Cλ) | (° C.) | (° C.) | (° C.) | (° C.) | (° C.) ± S.D. |
| Control #1 | WT | WT | 67.6 | 68.5 | 72.1 | 79.8 | 49.5 ± 0.7 |
| Control #2 | S183K/V133E | WT | 64.8 | 68.7 | NA | 79.3 | 48.4 ± 0.3 |
| Variant #33 | S183K/V133E | A141D/T117R | 64.5 | 68.8 | NA | 79.2 | 49.3 ± 1.4 |
| Variant #34 | S183K/V133E | A141E/T117R | 63.7 | 68.7 | NA | 79.4 | 50.3 ± 1.1 |
| Variant #35 | S183K/V133E | A141S/T117R | 64 | 68.7 | NA | 79.4 | 48.3 ± 0.5 |
| Variant #36 | S183K/V133E | A141T/T117R | 64.5 | 68.7 | NA | 79.4 | 52.5 ± 0.3 |
| Variant #41 | S183K/V133E | A141D/T117K | 64.5 | 68.7 | NA | 79.3 | 50.8 ± 0.3 |

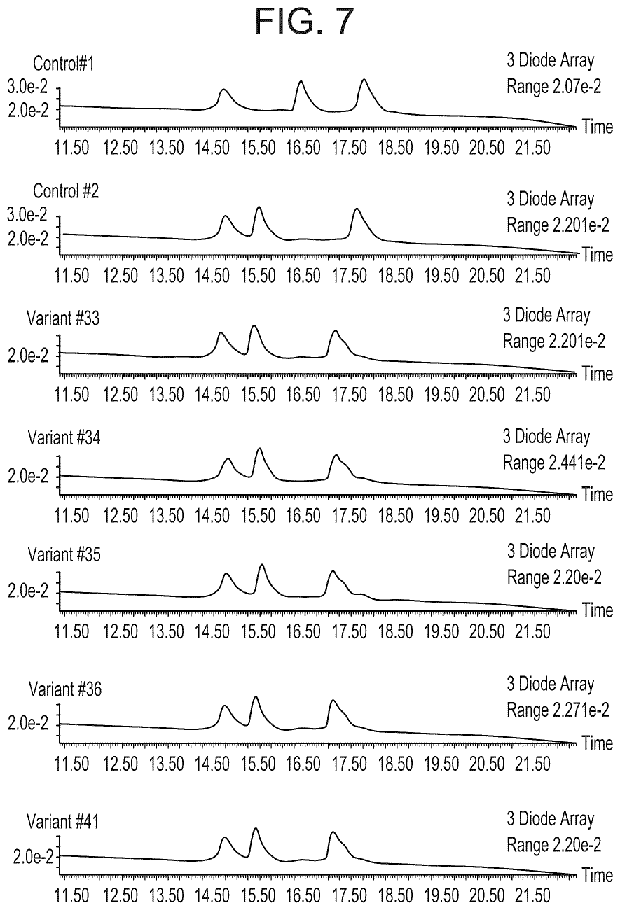

FIG. 7 and Table 8 show the sub-unit mass spectrum data of Antigen 1/Antigen 2 DuetMabs carrying the selected charge pair variants. The alignment of theoretical mass and measured mass confirmed molecule integrity and LC/HC association identity of each variant.

TABLE 8
MS results of sub-unit LC/MS analysis.

| | Theoretical mass | Measured mass |
|---|---|---|
| Control #1 | | |
| Antigen 1_LC + Antigen 1 | 47474 | 47474 |
| HC Fab | | |
| Antigen 2_LC + Antigen 2 | 47020 | 47021 |
| HC Fab | | |
| Fc with 2 G0F | 53015 | 53016 |
| Control #2 | | |
| Antigen 1_LC + Antigen 1 | 47545 | 47545 |
| HC Fab | | |
| Antigen 2_LC + Antigen 2 | 47020 | 47021 |
| HC Fab | | |
| Fc with 2 G0F | 53015 | 53016 |
| Variant #33 | | |
| Antigen 1_LC + Antigen 1 | 47545 | 47545 |
| HC Fab | | |
| Antigen 2_LC + Antigen 2 | 47119 | 47120 |
| HC Fab | | |
| Fc with 2 G0F | 53015 | 53016 |
| Variant #34 | | |
| Antigen 1_LC + Antigen 1 | 47545 | 47545 |
| HC Fab | | |
| Antigen 2_LC + Antigen 2 | 47133 | 47134 |
| HC Fab | | |
| Fc with 2 G0F | 53015 | 53016 |
| Variant #35 | | |
| Antigen 1_LC + Antigen 1 | 47545 | 47545 |
| HC Fab | | |
| Antigen 2_LC + Antigen 2 | 47091 | 47092 |
| HC Fab | | |
| Fc with 2 G0F | 53015 | 53016 |
| Variant #36 | | |
| Antigen 1_LC + Antigen 1 | 47545 | 47545 |
| HC Fab | | |
| Antigen 2_LC + Antigen 2 | 47105 | 47106 |
| HC Fab | | |
| Fc with 2 G0F | 53015 | 53016 |
| Variant #41 | | |
| Antigen 1_LC + Antigen 1 | 47545 | 47545 |
| HC Fab | | |
| Antigen 2_LC + Antigen 2 | 47091 | 47092 |
| HC Fab | | |
| Fc with 2 G0F | 53015 | 53016 |

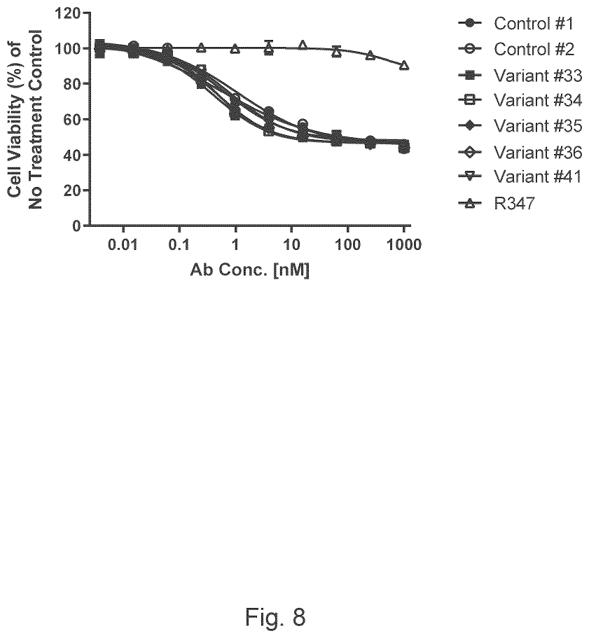

FIG. 8 shows the cytotoxicity properties of Antigen 1/Antigen 2 DuetMabs carrying the selected charge pair variants, as determined by quantification of ATP, which signals the presence of metabolically active cells. The variants #33, #34, #35, #36, and #41 displayed the cytotoxicity to the same extent of controls #1 and #2, suggesting the charge pair variants did not impact the biological function of Antigen 1/Antigen 2 DuetMab.

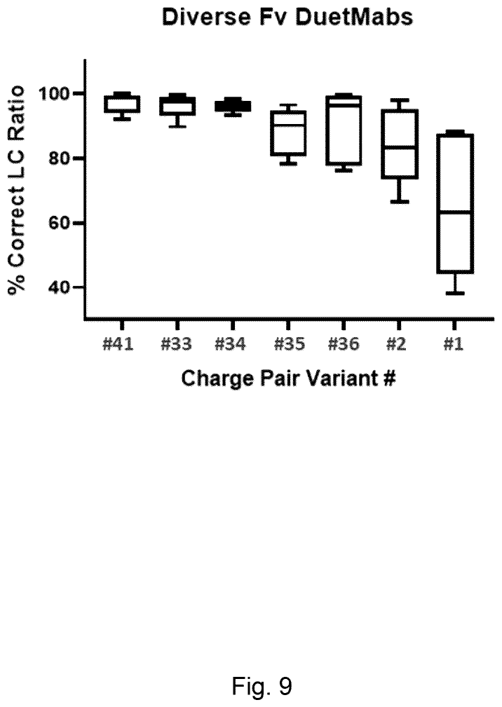

FIG. 9 and Table 9 summarize the expression and biochemical profiles of selected charge pair variants in diverse Fv DuetMabs. FIG. 9 shows the correct LC ratio data of Table 9 plotted in grouped box chart. Charge pair variants #33, #34, #35, #36, and #41 showed improved correct LC ratio compared to controls #1 and #2 among different Fv DuetMabs.

TABLE 9
Summary of expression and biochemical profiles of selected
charge pair variants in diverse Fv DuetMabs.

| | | Hole arm | | Knob arm | | Day 10/12 | % Correct | % |
|---|---|---|---|---|---|---|---|---|
| DuetMab | Sample # | CH1 | Cκ | CH1 (V12) | Cλ (V12) | Titer (μg/mL) | LC Ratio | Monomer |
| Antigen 1/ | 1 | WT | WT | WT | WT | 228.4 | 63.3 | 91.8 |
| Antigen 2 | 2 | S183K | V133E | WT | WT | 189.2 | 92.1 | 91.7 |
| | 33 | S183K | V133E | A141D | T117R | 190.2 | 98.6 | 93.5 |
| | 34 | S183K | V133E | A141E | T117R | 218.3 | 97.1 | 92.3 |
| | 35 | S183K | V133E | A141S | T117R | 237.0 | 90.2 | 94.2 |
| | 36 | S183K | V133E | A141T | T117R | 247.1 | 99.7 | 92.5 |
| | 41 | S183K | V133E | A141D | T117K | 82.2 | 96.3 | 93.5 |
| Antigen 1/ | 1 | WT | WT | WT | WT | 364.2 | 38.2 | 94.8 |
| Antigen 3 | 2 | S183K | V133E | WT | WT | 313.6 | 66.4 | 97.3 |
| | 33 | S183K | V133E | A141D | T117R | 289.9 | 89.8 | 96.5 |
| | 34 | S183K | V133E | A141E | T117R | 365.5 | 93.6 | 96.2 |
| | 35 | S183K | V133E | A141S | T117R | 262.7 | 78.4 | 97.4 |
| | 36 | S183K | V133E | A141T | T117R | 241.1 | 79.2 | 97.1 |
| | 41 | S183K | V133E | A141D | T117K | 252.7 | 92.1 | 97.1 |
| Antigen 4/ | 1 | WT | WT | WT | WT | 254.9 | 50.2 | 100.0 |
| Isotype | 2 | S183K | V133E | WT | WT | 243.2 | 80.7 | 100.0 |
| Control | 33 | S183K | V133E | A141D | T117R | 173.2 | 99.7 | 100.0 |
| | 34 | S183K | V133E | A141E | T117R | 305.4 | 98.2 | 100.0 |
| | 35 | S183K | V133E | A141S | T117R | 257.0 | 96.5 | 100.0 |
| | 36 | S183K | V133E | A141T | T117R | 219.5 | 98.9 | 100.0 |
| | 41 | S183K | V133E | A141D | T117K | 175.4 | 98.6 | 100.0 |
| Antigen 5/ | 1 | WT | WT | WT | WT | 162.2 | 86.9 | 95.4 |
| Antigen 3 | 2 | S183K | V133E | WT | WT | 124.8 | 98.1 | 100.0 |
| | 33 | S183K | V133E | A141D | T117R | 93.8 | 96.8 | 100.0 |
| | 34 | S183K | V133E | A141E | T117R | 90.5 | 96.0 | 100.0 |
| | 35 | S183K | V133E | A141S | T117R | 124.0 | 92.9 | 100.0 |
| | 36 | S183K | V133E | A141T | T117R | 127.0 | 96.3 | 100.0 |
| | 41 | S183K | V133E | A141D | T117K | 100.5 | 100.0 | 100.0 |
| Antigen 6/ | 1 | WT | WT | WT | WT | 138.7 | 88.2 | 96.9 |
| Antigen 1 | 2 | S183K | V133E | WT | WT | 110.6 | 83.4 | 99.0 |
| | 33 | S183K | V133E | A141D | T117R | 128.7 | 97.7 | 96.5 |
| | 34 | S183K | V133E | A141E | T117R | 63.1 | 95.2 | 98.9 |
| | 35 | S183K | V133E | A141S | T117R | 143.4 | 82.6 | 95.0 |
| | 36 | S183K | V133E | A141T | T117R | 109.3 | 76.1 | 96.6 |
| | 41 | S183K | V133E | A141D | T117K | 124.4 | 98.7 | 97.8 |

### Example 4—Crystallographic Investigation of Proposed Mutations in CH1-CL (Lambda) Interface

X-ray crystallography was carried out in order to further investigate the lambda charge variants at the light chain: CH1 interface.

### Fab Cloning and Expression

The coding sequences for (i) the variable domain of a light chain from an anti-Antigen 2 antibody and the constant domain of human lambda light chain containing T117R, S122C, and C212V mutations, and (ii) the variable domain of an anti-Antigen 2 antibody heavy chain and CH1 domain containing A141 D or A141 E as well as F126C and C220V mutations were ordered as synthetic DNA gBlocks from Integrated DNA Technologies (Coralville, IA). The coding sequence of light chain was flanked by N-terminal BssHII and C-terminal NheI restriction sites, and the heavy chain was flanked by N-terminal BsrGI and C-terminal EcoRI restriction sites to facilitate cloning. The gBlocks were digested and inserted into a mammalian expression vector (pOE; AstraZeneca, Gaithersburg, MD). One Shot Top10 chemically competent Escherichia coli cells (Invitrogen, Carlsbad, CA) were used as the host for gene cloning.

Both Fabs were transiently expressed in a suspension of human embryonic kidney (HEK) 293 cells, using 293fectin Transfection Reagent (Life Technologies, Carlsbad, CA) and standard protocols. Cells were grown in FreeStyle 293-F Expression Medium (Life Technologies) for 10 days and fed with a proprietary cell feed solution (AstraZeneca), after which the suspension was spun down and the supernatant filtered through a 0.2 μM filter. The Fab was purified from the supernatant using a 5 ml CaptureSelect CH1-XL column (Thermo Fisher Scientific, Waltham, MA), dialyzed against 25 mM Hepes pH 7 and further polished with a 5 ml HiTrap SP HP cation exchange column (Cytiva, Marlborough, MA) in a NaCl gradient in order to improve the homogeneity of the sample.

### Crystallization, Harvesting and X-Ray Diffraction Data Collection

The Fabs were individually run on a Superdex 200 Increase 10/300 GL column (Cytiva) pre-equilibrated with 25 mM HEPES, pH 7.5 and 100 mM NaCl to ensure homogeneity of the samples before setting up crystallization screens. Initial crystallization trials for both proteins were carried out by the sitting-drop vapor-diffusion method at 20° C. The crystallization drops were dispensed in 96-well crystallization plates (Intelli-Plate 102-0001-20; Art Robbins Instruments, Sunnyvale, CA) using a Phoenix crystallization robot (Art Robbins Instruments) and commercially available crystallization screens. The drops were composed of equal volumes of protein and reservoir buffer.

### Results

Diffraction quality crystals were harvested directly from the original sitting drop plates from the following crystallization solutions: A141 E: 0.1 M BIS-TRIS pH 6.5; 25% w/v PEG 3350 at a protein concentration of 18.4 mg/ml. A141 D: 200 mM sodium chloride; 0.1 M BIS-TRIS pH 5.5; 25% w/v PEG 3350 at a protein concentration of 9 mg/ml. All crystals harvested for X-ray analysis were flash-cooled in liquid nitrogen, and diffraction experiments were performed on a beamline B114-1 at Stanford Synchrotron Radiation Lightsource (Menlo Park, CA) at 100K. Diffraction data collected from a single crystal for each Fab were processed, integrated, and scaled with XDS software (Kabsch, 2010).

Structures of both Fab molecules were determined using molecular replacement method with program MolRep (Vagin, 1997) from CCP4 (Winn, 2011) suite of crystallographic software. Model building was performed using Coot (Emsley, 2004), for refinement program Refmac5 (Kovalevskiy, 2018) was used.

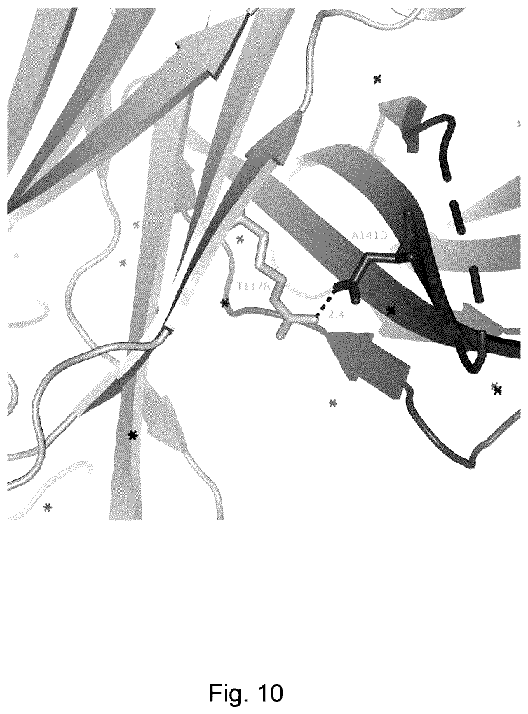

Crystal of T117R/A141 D Fab diffracted to 2.1 Å. Upon completion of the refinement we found that in line with our prediction side chains of mutated amino acids indeed established quite strong hydrogen bond (FIG. 10).

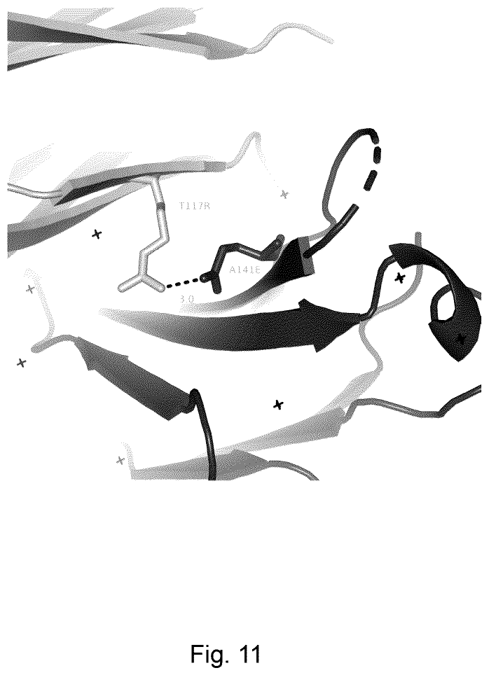

Crystal of T117R/A141 E Fab diffracted to 2.0 Å. Upon completion of the refinement we found that in line with our prediction side chains of mutated amino acids indeed established hydrogen bond (FIG. 11).

### Example 5—Generation of 2+1 Bispecific Antibodies Containing Lambda Charge Pairs

The materials and methods set forth herein were used to perform the experiments described in subsequent examples. All reagents were from Thermo Fisher Scientific, Waltham, MA, unless stated otherwise.

Construction of p2+1-Heavy mammalian expression vectors for Duet2 (2+1) Bispecific with charge mutations

Vector p2+1-Heavy was constructed on the backbone of pDuet-Heavy described in Example 2. For construction of p2+1-Heavy vector with charge mutations, the “Hole” heavy chain was cloned into the vector by BssHII/HindII as previously described. The “Knob” heavy chain was cloned into the vector by a synthesized DNA fragment of VH-CH1-VH-CH1-CH2-CH3 domains using restriction cloning technique by BsrGI/EcoRI, where the preceding VH-CH1 segment corresponded to the sequence found on the “Hole” heavy chain, and the subsequent VH-CH1 segment contained the VH against another target as well as the charge mutation A141 D and V12 DS in CH1. The pDuet-Light vector is common for both the Duet2 (2+1) Bispecific and DuetMab constructs.

### Expression, Affinity Purification and Protein Quantification

All Duet2 (2+1) Bispecific constructs were transiently expressed and purified as described above for DuetMab molecules.

Binding kinetics assays, Accelerated stability studies, Subunit LC-MS analysis, and Differential scanning calorimetry analysis (DSC) were conducted essentially as described above for DuetMab proteins.

xCELLigence Cell Killing Assay

T cell-mediated cytotoxicity was assessed with the xCELLigence Real-Time Cell Analyzer (ACEA Biosciences). A total of 1×10⁴ target cells per well, resuspended with RPM11640 media supplied with 10% heat-inactivated FBS and 50 μM of 2-Mercaptoethanol, were seeded in E-Plate. After overnight cell adherence at 37° C. and 5% C02, effector cells (PBMCs from healthy donor) were added at E:T ratio of 10:1 and antibodies were added at various concentrations. Cell index (i.e., relative cell impedance) values were monitored every 10 minutes and the time point closer to 48 hours (above or below) was used for data analysis. Cell cytotoxicity was normalized to the maximal cell index value without antibody treatment and plotted using GraphPad Prism v 9.0.0.

### CD69 and CD25 Expression Assay

Flow cytometry was used to characterize human T-cell activation in T cell-mediated cytotoxicity assay. After two days of incubation, T cells from cytotoxicity assay were harvested and analyzed. T cells from PBMCs were measured by staining for surface expression of CD2 and CD4 (both from BioLegend, San Diego, CA, USA). T cell activation was determined by staining of both T cell activation markers (CD69, CD25) (both from BioLegend, San Diego, CA, USA) and analyzed by flow cytometer FACSymphony A3 from BD. Data was analyzed with FlowJo v 10.6.1 and plotted using GraphPad Prism v 9.0.0.

### Results

Table 10 summarizes the expression and biochemical profiles of Antigen 1/CD3 and NIP228/CD3 Duet2 (2+1) Bispecific molecules carrying the selected sets of charge pairs produced in 500 mL cell culture. For additional analysis, the DuetMabs were further purified by light chain affinity chromatography to remove mispaired byproducts, and aggregates were removed by preparative SEC.

TABLE 10
Production of Duet2 (2 + 1) Bispecific with charge mutations.

| | | | | | | | | | Profiles after protein | | Profiles of after | | |
|---|---|---|---|---|---|---|---|---|---|---|---|---|---|
| | | | | | CD3 (Knob | | Titer | | A purification | | Light chain purification | | |
| Duet2 | Antigen 1/NIP228 | | Antigen 1/NIP228 | | arm) | | (ug/mL) | | Ratio of | | Ratio of | | |
| (2+1) | (Hole arm) | | (Knob arm) | | CH1 | Cλ | Day | Day | Kappa and | % | Kappa and | % | Endotoxin |
| Bispecific | CH1 | C_{κ} | CH1 | C_{κ} | (V12) | (v12) | 7 | 14 | Lambda | Monomer | Lambda | Monomer | (EU/mg) |
| Antigen 1/ | S183K | V133E | S183K | V133E | A141D | T117R | 55 | 130 | 2.49:1 | 90 | 2:1 | >99 | <0.5 |
| CD3 2 + 1 | | | | | | | | | | | | | |
| NIP228/ | S183K | V133E | S183K | V133E | A141D | T117R | 96 | 380 | 2.39:1 | 91 | 1.9:1 | >99 | <0.5 |
| CD3 2 + 1 | | | | | | | | | | | | | |

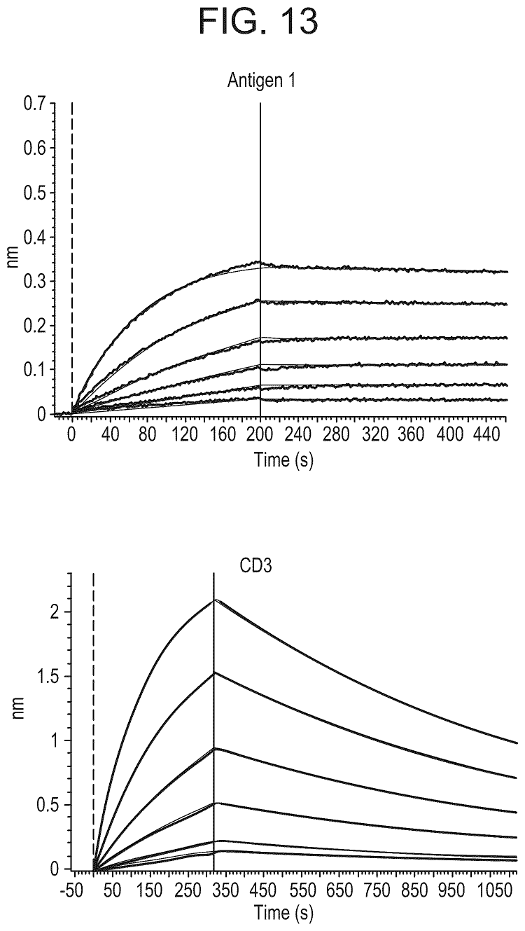

FIG. 13 shows binding kinetics of Antigen 1/CD3 Duet2 (2+1) Bispecific molecule to Antigen 1 and CD3 antigens. Table 11 shows the response signals and fitting curves.

TABLE 11
Kinetic Binding of Antigen 1/CD3 Duet2 (2 + 1) Bispecific with Charge Mutations.

| | | | Antigen 1 (CH1/Cκ) − | | | |
|---|---|---|---|---|---|---|
| | | | CD3 (CH1/Cλ) | | | |
| | | Antigen 1 | Antigen 1 | CD3 | | |
| Duet2 (2 + 1) Bispecific | Antigen | (CH1/Cκ) | (CH1/Cκ) | (CH1/Cλ) | KD (M) | KD Error |
| Antigen 1/CD3 2 + 1 | Antigen 1 | S183K/V133E | S183K/V133E | A141D/T117R | 1.521E−10 | 2.291E−12 |
| Antigen 1/CD3 2 + 1 | CD3 | S183K/V133E | S183K/V133E | A141D/T117R | 3.180E−08 | 2.752E−11 |

| Duet2 (2 + 1) Bispecific | ka (1/Ms) | ka Error | kdis (1/s) | kdis Error | Full X\hat{ }2 | Full R\hat{ }2 |
|---|---|---|---|---|---|---|
| Antigen 1/CD3 2 + 1 | 5.884E05 | 1.177E03 | 8.948E−05 | 1.336E−06 | 0.2425 | 0.9987 |
| Antigen 1/CD3 2 + 1 | 3.004E04 | 2.455E01 | 9.555E−04 | 2.721E−07 | 2.4719 | 0.9997 |

Table 12 summarizes the thermal stabilities of Antigen 1/CD3 and NIP228/CD3 Duet2 (2+1) Bispecific molecules by differential scanning fluorimetry (DSF) and their accelerated stability profiles. The Duet2 (2+1) Bispecific molecules showed no concerns for aggregation or fragmentation after heat stress.

TABLE 12
Developability Summary of Duet2 (2+1) Bispecific with Charge Mutations

| | Accelerated Stability (45° C., 14 d) | | | | | |
|---|---|---|---|---|---|---|
| Duet2 (2 + 1) | Monomer | Aggregate | Fragment | ΔRT from | DSF | |
| Bispecific | Loss (%) | Increase (%) | Increase (%) | NIP228 (m) | Tₒₙₛₑₜ | Tₘ |
| Antigen 1/CD3 2 + 1 | 6.18 | 0.30 | 5.87 | 6.18 | 50 | 60.6 |
| NIP228/CD3 2 + 1 | 3.19 | 0.69 | 2.49 | 3.19 | 47 | 58.3 |

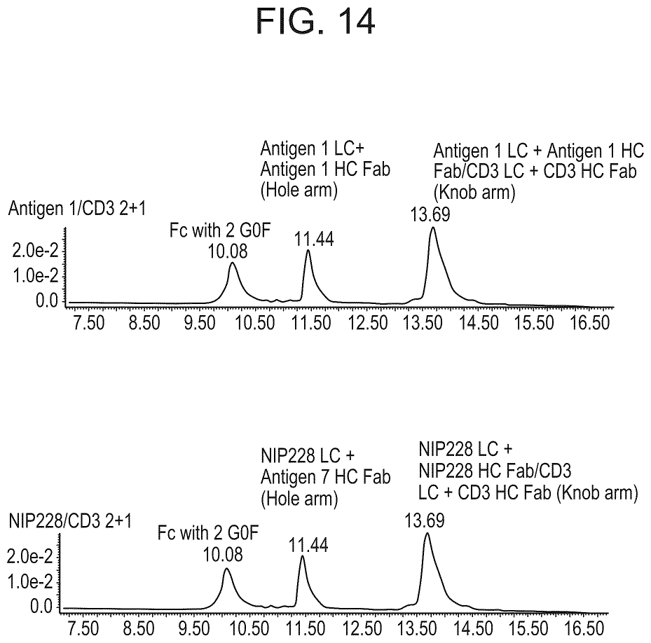

FIG. 14 and Table 13 show the sub-unit mass spectrum data of Antigen 1/CD3 and NIP228/CD3 Duet2 (2+1) Bispecific molecules. The alignment of theoretical mass and measured mass confirmed molecule integrity and LC/HC association identity of each variant.

TABLE 13
MS results of sub-unit LC/MS analysis.

| | measured | theoretical |
|---|---|---|
| | mass | mass |
| Antigen 1/CD3 2 + 1 | | |
| Fc with 2 G0F | 53095 | 53095 |
| Antigen 1 LC + Antigen 1 HC Fab (Hole arm) | 47545 | 47544 |
| Antigen 1 LC + Antigen 1 HC Fab + CD3 LC + | 95876 | 95876 |
| CD3 HC Fab (Knob arm) | | |
| NIP228/CD3 2 + 1 | | |
| Fc with 2 G0F | 53095 | 53095 |
| NIP228-LC + NIP228 HC Fab (Hole arm) | 46991 | 46991 |
| NIP228 LC + NIP228 HC Fab + CD3 LC + | 95322 | 95322 |
| CD3 HC Fab (Knob arm) | | |

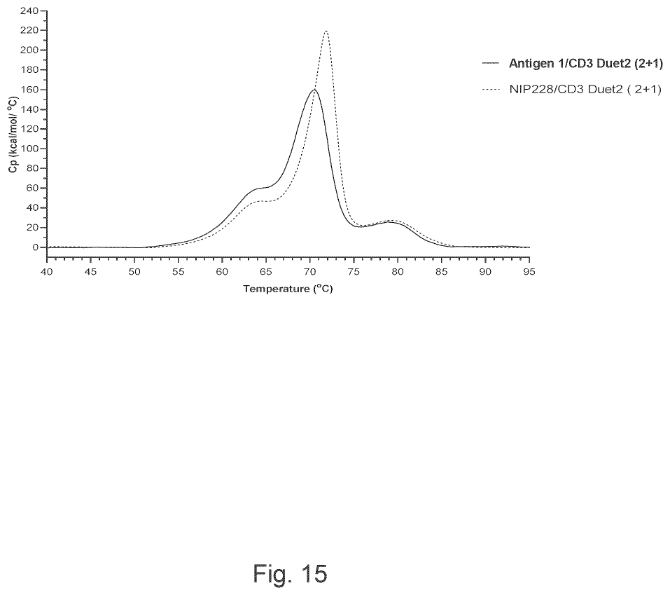

FIG. 15 and Table 14 show the thermal stability studies of Antigen 1/CD3 and NIP228/CD3 Duet2 (2+1) Bispecific molecules using differential scanning calorimetry (DSC) analysis. FIG. 15 illustrates the stacked thermograms for Antigen 1/CD3 and NIP228/CD3 Duet2 (2+1) Bispecific molecules. Deconvolution of the thermograms revealed the transitions for the Fab, CH2, and CH3 domains, where some transitions were overlapped and under the same TM peaks. Table 14 lists the deconvoluted TM and approximated Tₒₙₛₑₜ values of Duet2 (2+1) Bispecific molecules.

TABLE 14
MS results of sub-unit LC/MS analysis.

| | T_{M1} | T_{M2} | T_{M3} | Approximated Tₒₙₛₑₜ |
|---|---|---|---|---|
| Name | (° C.) | (° C.) | (° C.) | (° C.) ± S.D. |
| Antigen 1/CD3 Duet2 (2:1) | 64.6 | 70.5 | 79.0 | 51.8 ± 0.4 |
| NIP228/CD3 Duet2 (2:1) | 66.7 | 71.8 | 79.3 | 53 ± 1.3 |

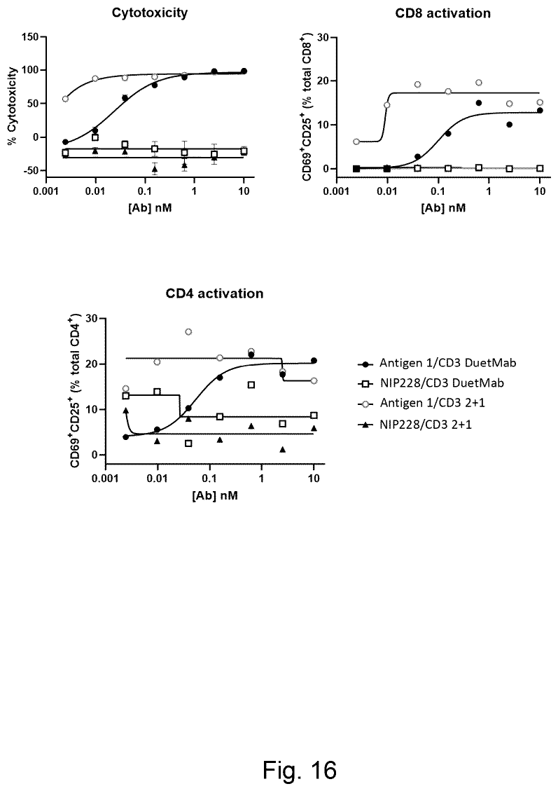

FIG. 16 shows the cytotoxicity properties of DuetMab and Duet2 (2+1) bispecific molecules, as determined by xCELLigence cytotoxicity assay. Antigen 1/CD3 Duet2 bispecific with two anti-Antigen 1 Fab arms exhibited superior potency in eliminating the Antigen 1-expressing target cells compared to corresponding 1+1 Antigen 1/CD3 DuetMab. Antigen 1/CD3 Duet2 bispecific treated wells exhibited elevated CD8 and CD4 T cell activations compared to wells treated with Antigen 1/CD3 DuetMab. NIP228/CD3 DuetMab and NIP228/CD3 Duet2 (2+1) bispecifics were included in this assay as isotype controls. The control molecules exhibited low cytotoxicity and induced limited CD8 and CD4 T cell activations.

### Sequences

1. Amino acid sequence of a WT CLλ constant region (SEQ ID NO: 1)

GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTECS

2. Amino acid sequence of a ‘V12’ LC lambda constant (CLλ) region modified to form an engineered disulfide bridge (SEQ ID NO: 2)

Following substitutions are underlined:
Engineered disulfide: S122C, C212V

GQPKAAPSVTLFPPCSEELQANKATLVCLISDFYPGAVTVAWKADSSPVK
AGVETTTPSKQSNNKYAASSYLSLTPEQWKSHRSYSCQVTHEGSTVEKTV
APTEVS

3. Amino acid sequence of a WT LC kappa constant (Cκ) region (SEQ ID NO: 3)

RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

4. Amino acid sequence of a IgG1 CH1 (SEQ ID NO:4)

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSC

5. Amino acid sequence of a ‘V12’ CH1 modified to form an engineered disulfide bridge (SEQ ID NO:5)

Following substitutions are underlined:
Engineered disulfide: F126C, C220V

ASTKGPSVCPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSV

6. Amino acid sequence of an IgG1 CH2 (SEQ ID NO:6)

LLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIE
KTIS

7. Amino acid sequence of an IgG1 CH3 (SEQ ID NO:7)

GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

8. Amino acid sequence of an IgG1 heavy chain polypeptide (SEQ ID NO:8)

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

9. Amino acid sequence of an IgG1 CH3 engineered to contain a “hole” mutation (SEQ ID NO:9)

Following substitutions are underlined:
“Hole” mutations (T366S, L368A, and Y407V).

GQPREPQVYTLPPSREEMTKNQVSLSCAVKGFYPSDIAVEWESNGQPENN
YKTTPPVLDSDGSFFLVSKLTVDKSRWQQGNVFSCSVMHEALHNHYTQKS
LSLSPGK

10. Amino acid sequence of an IgG1 CH3 engineered to contain a stabilizing cysteine and a “hole” mutation (SEQ ID NO:10)

Following substitutions are underlined:
Hole” mutations (T366S, L368A, and Y407V); and stabilizing cysteine mutation (Y349C).

GQPREPQVCTLPPSREEMTKNQVSLSCAVKGFYPSDIAVEWESNGQPENN
YKTTPPVLDSDGSFFLVSKLTVDKSRWQQGNVFSCSVMHEALHNHYTQKS
LSLSPGK

11. Amino acid sequence of an IgG1 CH3 engineered to contain a “knob” mutation (SEQ ID NO:11)

Following substitutions are underlined:
“Knob” mutation (T366W).

GQPREPQVYTLPPSREEMTKNQVSLWCLVKGFYPSDIAVEWESNGQPENN
YKTTPPVLDSDGSFFLYSKLTVDKSRWQQGNVFSCSVMHEALHNHYTQKS
LSLSPGK

12. Amino acid sequence of an IgG1 CH3 engineered to contain a stabilizing cysteine and a “knob” mutation (SEQ ID NO:12)

Following substitutions are underlined:
“Knob” mutation (T366W); stabilizing cysteine mutation (S354C).

GQPREPQVYTLPPCREEMTKNQVSLWCLVKGFYPSDIAVEWESNGQPENN
YKTTPPVLDSDGSFFLYSKLTVDKSRWQQGNVFSCSVMHEALHNHYTQKS
LSLSPGK

13. Amino acid sequence of an IgG1 heavy chain polypeptide engineered to contain a stabilizing cysteine and “hole” mutations (SEQ ID NO:13)

Following substitutions are underlined:
Hole” mutations (T366S, L368A, and Y407V); and stabilizing cysteine mutation (Y349C).

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVCTLPPSREEMTKNQVSLSC
AVKGFYPSDIAVEWESNGQPENNYKTTPPVLDSDGSFFLVSKLTVDKSRW
QQGNVFSCSVMHEALHNHYTQKSLSLSPGK

14. Amino acid sequence of an IgG1 heavy chain polypeptide engineered to contain a stabilizing cysteine and “knob” mutations (SEQ ID NO:14)

Following substitutions are underlined:
“Knob” mutation (T366W); stabilizing cysteine mutation (S354C).

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRW
QQGNVFSCSVMHEALHNHYTQKSLSLSPGK

15. Amino acid sequence of a ‘V12’ IgG1 heavy chain polypeptide engineered to contain a stabilizing cysteine, interchain cysteine mutations, and “knob” mutations (SEQ ID NO:15) Following substitutions are underlined:

“Knob” mutation (T366W); interchain cysteine mutations (F126C and C220V); stabilizing cysteine mutation (S354C).

ASTKGPSVCPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSVDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSRW
QQGNVFSCSVMHEALHNHYTQKSLSLSPGK

16. Linker (SEQ ID NO:16)

GGGGS

17. Linker (SEQ ID NO:17)

SGGGGS

18. Linker (SEQ ID NO:18)

GGGGSGGGGS

1. A multispecific antibody comprising:
(a) a first antigen binding arm comprising a first light chain that is disulfide linked to a first heavy chain constant region 1 (CH1); and
(b) a second antigen binding arm comprising a second light chain that is disulfide linked to a second CH1; and
(c) a third antigen binding arm comprising a third light chain that is disulfide linked to a third CH1,
wherein the first antigen binding arm binds to a first epitope, and the second and the third antigen binding arms bind to a second epitope,
wherein the third antigen binding arm is fused to the first or second antigen binding arm,
wherein the first antigen binding arm, or both the second and the third antigen binding arms, comprise a lambda charge pair located at one or more of the following pairs of positions in a constant light chain lambda region (CLU) of the light chain and the CH1:
i. position 117 in the CLλ and position 141 in the CH1;
ii. position 117 in the CLλ and position 185 in the CH1;
iii. position 119 in the CLX and position 128 in the CH1;
iv. position 134 in the CLλ and position 128 in the CH1;
v. position 134 in the CLλ and position 145 in the CH1;
vi. position 134 in the CLλ and position 183 in the CH1;
vii. position 136 in the CLλ and position 185 in the CH1;
viii. position 178 in the CLλ and position 173 in the CH1; and
ix. position 117 in the CLλ and position 187 in the CH1,
wherein the lambda charge pair comprises a positively charged amino acid residue optionally selected from arginine, lysine or histidine located at one of the positions in the lambda charge pair and a negatively charged amino acid residue optionally selected from aspartic acid, glutamic acid, serine or threonine located at the other position in the lambda charge pair, and
wherein the numbering is according to the EU index.

2. The multispecific antibody according to claim 1, wherein the lambda charge pair is located at position 117 in the CLU and position 141 in the CH1.

3. (canceled)

4. The multispecific antibody according to claim 2, wherein the lambda charge pair is selected from the following list:
b. arginine at position 117 of the CLU and aspartic acid at position 141 of the CH1;
c. arginine at position 117 of the CLU and glutamic acid at position 141 of the CH1;
d. arginine at position 117 of the CLU and serine at position 141 of the CH1;
e. arginine at position 117 of the CLU and threonine at position 141 of the CH1; and
f. lysine at position 117 of the CLU and aspartic acid at position 141 of the CH1.

5-8. (canceled)

9. The multispecific antibody according to claim 1, wherein the lambda charge pair is located at position 134 in the CLU and position 183 in the CH1, optionally wherein the lambda charge pair is a lysine at position 134 of the CLX, and an aspartic acid or a serine at position 183 of the CH1.

10-11. (canceled)

12. The multispecific antibody according to claim 9, wherein either:
(i) the disulfide link between the first light chain and first CH1 is formed between a pair of cysteines engineered into the first light chain and first CH1, and the disulfide link between both the second light chain and second CH1, and third light chain and third CH1, is formed between a pair of native cysteines; or
(ii) the disulfide link between both the second light chain and second CH1, and third light chain and third CH1, is formed between a pair of cysteines engineered into both the second light chain and the second CH1, and third light chain and third CH1, and the disulfide link between the first light chain and first CH1 is formed between a pair of native cysteines.

13. The multispecific antibody according to claim 12, wherein the pair of cysteines engineered into light chain and CH1 are located at position 122 of the light chain and position 126 of the CH1, and wherein the light chain comprises a non-cysteine residue at position 212 and the CH1 comprises a non-cysteine residue at position 220, optionally wherein the non-cysteine residues are valines.

14. The multispecific antibody according to claim 9, wherein the CLU comprises an amino acid sequence having at least 90%, 91%, 92%, 93%, 94%, 95%, 96%, 97%, 98%, or 99% identity to SEQ ID NO: 1 or SEQ ID NO: 2.

15. The multispecific antibody according to claim 9, wherein the first, second and/or third CH1 comprises an amino acid sequence having at least 90%, 91%, 92%, 93%, 94%, 95%, 96%, 97%, 98%, or 99% identity to SEQ ID NO: 4 or SEQ ID NO: 5.

16. (canceled)

17. The multispecific antibody according to claim 9, wherein the antigen binding arm(s) comprising the CLκ comprises a kappa charge pair located in the CLκ and CH1 of the antigen binding arm(s), and wherein the negatively charged amino acid residue in the kappa charge pair is at position 133 of the CLκ, and the positively charged amino acid residue in the kappa charge pair is at position 183 of the CH1,
optionally wherein the negatively charged amino acid residue at position 133 of the CLκ is a glutamic acid, and wherein the positively charged amino acid residue at position 183 of the CH1 is a lysine.

18. (canceled)

19. The multispecific antibody according to claim 17, wherein the CLκ comprises an amino acid sequence having at least 90%, 91%, 92%, 93%, 94%, 95%, 96%, 97%, 98%, or 99% identity to SEQ ID NO: 3.

20. A multispecific antibody comprising:
(a) a first antigen binding arm comprising a first light chain that is disulfide linked to a first heavy chain constant region 1 (CH1), the first light chain comprising a constant light chain lambda region (CLU), wherein:
(i) the first antigen binding arm comprises a lambda charge pair located at position 117 in the CLU and position 141 in the first CH1, wherein the lambda charge pair comprises a positively charged amino acid residue selected from arginine, lysine or histidine located at one of the positions in the lambda charge pair and a negatively charged amino acid residue selected from aspartic acid, glutamic acid, serine or threonine located at the other position in the lambda charge pair; and
(ii) the disulfide link between the first light chain and first CH1 is formed between a pair of cysteines engineered into the CLU and first CH1; and
(b) a second antigen binding arm comprising a second light chain that is disulfide linked to a second CH1; and
(c) a third antigen binding arm comprising a third light chain that is disulfide linked to a third CH1, wherein:
i. the second and third light chains comprise a constant light chain kappa region (CLκ); and
ii. the second and the third antigen binding arm both comprise a kappa charge pair located in the CLκ and corresponding CH1, wherein the kappa charge pair comprises a positively charged amino acid residue selected from arginine, lysine or histidine located at one of the positions in the kappa charge pair and a negatively charged amino acid residue selected from aspartic acid, glutamic acid, serine or threonine located at the other position in the kappa charge pair; and
iii. the disulfide link between the second light chain and second CH1 and the third light chain and third CH1 is formed between a pair of native cysteines in the CLκ and second CH1,
wherein the first antigen binding arm binds to a first epitope, and the second and third antigen binding arms bind to a second epitope,
wherein the third antigen binding arm is fused to the first or second antigen binding arm, and
wherein the numbering is according to the EU index.

21. The multispecific antibody according to claim 20, further comprising a fourth antigen binding arm comprising a fourth light chain that is disulfide linked to a fourth CH1, wherein the fourth antigen binding arm binds to the first epitope,
wherein the first and fourth antigen binding arms both comprise the lambda charge pair, and wherein:
(a) the third antigen binding arm is fused to the first antigen binding arm and the fourth antigen binding arm is fused to the second antigen binding arm; or
(b) the third antigen binding arm is fused to the second antigen binding arm and the fourth antigen binding arm is fused to the first antigen binding arm.

22. (canceled)

23. The multispecific antibody according to claim 21, wherein:
(a) the third antigen binding arm is fused to the N-terminus of the first antigen binding arm, and the second antigen binding arm is fused to the N-terminus of the fourth antigen binding arm; or
(b) the third antigen binding arm is fused to the C-terminus of the first antigen binding arm, and the second antigen binding arm is fused to the C-terminus of the fourth antigen binding arm.

24. The multispecific antibody according to claim 21, wherein
(i) the second and third light chains are the same; and/or
(ii) the first and fourth light chains are the same.

25-28. (canceled)

29. The multispecific antibody according to claim 20, comprising modifications in the CD3 of the Fc regions, wherein a substitution to generate a knob is a substitution to tryptophan at position 366 and the substitution to generate a hole is a substitution to generate a hole is one or more of the following:
1. a substitution to valine at position 407;
2. a substitution to serine at position 366; and
i. a substitution to alanine at position 368.

30. The multispecific antibody according to claim 29, wherein the CH3 domain containing the protuberance (knob) comprises a cysteine at position 354 and the CH3 domain containing the cavity (hole) comprises a cysteine at position 349.

31. The multispecific antibody according to 29, wherein at least one of the Fc regions comprises the amino acid substitutions:
(a) L234F/L235E/P331S;
(b) E233P/L234V/L235A/G236del/S267K; and/or
(c) M252Y/S254T/T256E.

32. The multispecific antibody according to claim 20, wherein the first antigen binding arm binds to an epitope on CD3.

33. The multispecific antibody according to claim 20, further comprising an additional antigen binding domain, wherein the additional antigen binding domain is a VHH, optionally wherein the VHH binds to an epitope on CD8.

34-37. (canceled)

38. One or more nucleic acid(s) encoding the multispecific antibody according to claim 1.

39. A vector comprising the nucleic acid(s) of claim 38.

40. An isolated host cell comprising the nucleic acid(s) of claim 39.

41. A pharmaceutical composition comprising the multispecific antibody according to claim 1.

42. A method of treating a disease in a patient in need thereof, the method comprising administering to the patient an effective amount of the multispecific antibody according to claim 1.

43. The method of claim 42, wherein the disease is cancer.

44. The multispecific antibody according to claim 1, for use as a medicament.

45. The multispecific antibody according to claim 1, for use in treating cancer.

46. Use of the multispecific antibody according to claim 1, for the manufacture of a medicament for the treatment of cancer.


SEQUENCE LISTING (18 sequences)

SEQ ID NO: 1 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 2 - protein, 106 aa, synthetic construct
GQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 3 - protein, 107 aa, Homo sapiens [sequence omitted]

SEQ ID NO: 4 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 5 - protein, 103 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSV

SEQ ID NO: 6 - protein, 104 aa, Homo sapiens
LLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTIS

SEQ ID NO: 7 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 107 aa, synthetic construct
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 107 aa, synthetic construct
GQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 107 aa, synthetic construct
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 107 aa, synthetic construct
GQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 330 aa, synthetic construct
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 5 aa, synthetic construct
GGGGS

SEQ ID NO: 17 - protein, 6 aa, synthetic construct
SGGGGS

SEQ ID NO: 18 - protein, 10 aa, synthetic construct
GGGGSGGGGS